(19) United States
(12) Patent Application Publication
(10) Pub. No.: US 2024/0408175 A1
(43) Pub. Date: Dec. 12, 2024

(54) BMP9 OR AN AGONIST THEREOF AND ITS USES RELATED TO REDUCING METASTASIS OF CANCER

(71) Applicant: THE UAB RESEARCH FOUNDATION, Birmingham, AL (US)

(72) Inventors: Mythreye Karthikeyan, Birmingham, AL (US); Zainab Shonibare, Birmingham, AL (US)

(73) Assignee: THE UAB RESEARCH FOUNDATION, Birmingham, AL (US)

(21) Appl. No.: 18/700,751

(22) PCT Filed: Oct. 18, 2022
(86) PCT No.: PCT/US2022/047037

§ 371 Date: Apr. 12, 2024

Related U.S. Application Data
(60) Provisional application No. 63/256,738, filed on Oct. 18, 2021.

(51) Int. Cl. A61K 38/18; A61K 31/337; A61K 33/243; A61P 35/04; C07K 14/51

(52) U.S. Cl. CPC A61K 38/1875; A61K 31/337; A61K 33/243; A61P 35/04; C07K 14/51

(57) ABSTRACT

Methods for using BMP9 or an agonist thereof to reduce metastasis of cancer are disclosed. The method may include administering an effective amount of BMP9 or an agonist thereof to a subject in need thereof, to reduce metastasis of cancer. The subject may be a human.

### PRIORITY CLAIM

This application claims priority to U.S. Provisional Application No. 63/256,738, filed on Oct. 18, 2021, the entire contents and disclosure of which are incorporated herein in their entirety.

### STATEMENTS REGARDING FEDERALLY FUNDED RESEARCH

This Invention was made with government support (NIHR01CA230628).

### FIELD OF THE INVENTION

The present invention relates to methods for reducing tumor growth and metastasis in a subject with cancer, the method comprising administering an effective amount of BMP9 or an agonist to the subject with cancer. In particular, the subject may have a gynecological cancer and the administration of BMP9 or an agonist may impair tumor growth.

### BACKGROUND

Gynecological cancers are ‘silent killer,’ and afflict more than 22,000 women annually in the United States alone. Due to its subtleness of symptoms, majority of patients are diagnosed when the cancer is at an advanced stage and thus, already spreading to other regions of the body which significantly impact patient outcome.

The metastatic cascade of gynecological cancers involves a series of steps which begins with the exfoliation of the tumor cells, directly shedding from the primary site into the peritoneum and omentum where they cluster to form spheroid-like aggregates 2,3, a phenomenon known as anchorage-independence. In the peritoneum, these aggregates disseminate throughout the abdominal cavity to potentially seed metastatic tumor growth and can also spread through circulation to reach the lungs.

Normal epithelial cells require extracellular matrix (ECM) attachment for survival and hence, detachment from the ECM as is the case in the exfoliated tumor cells initiates a cascade of cell death response, so-called anoikis.

Therefore, successful metastasis is dependent on the ability of the tumor cells to exhibit/acquire anoikis resistance. This underscores the need to investigate and identify how anoikis resistance can be targeted therapeutically, as restoration of anoikis sensitivity may help curb the migratory potential of metastatic tumors.

Bone morphogenetic proteins (BMPs) belong to the transforming growth factor-β (TGF-β) family. BMP9 is distinguished from other BMPs due to its unique receptor-conjugation specificity and its diverse roles in various processes within the cell. For example, BMP9 can inhibit hepatic glucose production, activate expression of several key enzymes of lipid metabolism, regulate endothelial cell growth and migration, induce apoptosis of prostate cancer cells. It is one of the most powerful BMPs inducing osteogenic differentiation and testicular bone formation.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim.

In some embodiments, the present disclosure is directed to a method for reducing metastasis in a subject with cancer, the method comprising administering an effective amount of BMP9 or an agonist thereof to the subject with cancer. The BMP9 may be recombinant BMP9 (rBMP9). The cancer may be selected from the group consisting of breast cancer, gynecological cancer, lung, neuroendocrine cancer, and combinations thereof. In some aspects, the cancer is a gynecological cancer, such as ovarian cancer. The BMP9 may be recombinant rBMP that is administered for a period of at least 7 days. The BMP9 may be rBMP and the BMP9 or an agonist thereof may be administered in an amount from 0.01 mg/kg to 50 mg/kg. The subject may be a mammal. In some aspects, the subject may be a human. Administering the BMP9 or an agonist thereof may suppress three dimensional spheroid cell invasion. The BMP9 or an agonist thereof may be administered in a pharmaceutical composition comprising the BMP9 or an agonist thereof and a pharmaceutically acceptable carrier. The pharmaceutical composition may further comprise an additional therapeutic agent. The additional therapeutic agent may be an anti-cancer compound. In some aspects, the anti-cancer compound is paclitaxel. In some aspects, the anti-cancer compound is cisplatin.

### BRIEF DESCRIPTION OF THE DRAWINGS

In the following detailed description, embodiments of the invention are described referring to the following figures (also referred to herein as FIG. or FIGS.):

FIG. 1A illustrates confocal microscopy images of OC cells cultured under anchorage independence for 48 hours and subsequently treated with vehicle (VEH) or 10 nM rhBMP2 or rhBMP9 for 24 hours in accordance with embodiments of the present disclosure.

FIG. 1B illustrates tumor luminescence images of NOD-SCID mice injected with PA1-luc-GFP cells with vehicle or rhBMP9 (5 mg/kg) administered i.p. daily in accordance with embodiments of the present disclosure.

FIG. 1C illustrates whole-animal luminescence quantified over time in accordance with embodiments of the present disclosure.

FIG. 1D is an image of omental tumor burden (left) and quantification of omental tumor weight (right) from mice that received vehicle or rhBMP9 injected with PA1-luc-GFP tumor cells in accordance with embodiments of the present disclosure.

FIG. 1E shows tumor luminescence images of NOD-SCID mice injected with SKOV3-luc-GFP cells with vehicle or rhBMP9 (5 mg/kg) administered i.p. daily in accordance with embodiments of the present disclosure.

FIG. 1F shows whole-animal luminescence quantified over time in accordance with embodiments of the present disclosure.

FIG. 1G shows a KM plot of SKOV3-Luc-GFP-injected mice receiving rhBMP9 compared with vehicle in accordance with embodiments of the present disclosure.

FIG. 1H shows H&E and TUNEL staining of PA1-luc-GFP tumors in accordance with embodiments of the present disclosure.

FIG. 1I shows H&E and TUNEL staining of SKOV3-luc-GFP tumors, in accordance with embodiments of the present disclosure.

FIG. 2A is a volcano plot of changes in gene expression in PA1 cells under anchorage-independence treated with vehicle or rhBMP9 for 24 h in accordance with embodiments of the present disclosure.

FIG. 2B shows a list of 15 top upregulated and downregulated genes in response to rhBMP9 from FIG. 2A in accordance with embodiments of the present disclosure.

FIG. 2C shows a Western blot (WB; top) and qRT-PCR (bottom) of SOX2 levels in a panel of OC cells in accordance with embodiments of the present disclosure.

FIG. 2D shows a qRT-PCR analysis of SOX2 mRNA levels in response to 24 hours of rhBMP9 (10 nM) treatment expressed relative to control untreated cells in accordance with embodiments of the present disclosure.

FIG. 2E shows a Western blot following treatment with rhBMP2, -4, and -9 and 10 (10 nM) or control for 24 hours in PA1 cells to assess SOX2 protein in accordance with embodiments of the present disclosure.

FIG. 2F shows a Western blot of rhBMP2 and rhBMP9 treatment and SOX2 protein in PA1 and OVCAR3 cells in accordance with embodiments of the present disclosure.

FIG. 2G shows a qRT-PCR analysis of relative SOX2 levels after rhBMP2 and rhBMP9 treatment normalized to untreated control in accordance with embodiments of the present disclosure.

FIG. 2H shows an immunohistochemistry (IHC) of SOX2 in PA1-Luc-GFP tumors from mice receiving vehicle or rhBMP9 from FIG. 1B in accordance with embodiments of the present disclosure.

FIG. 2I shows a qRT-PCR analysis of relative SOX2 transcript levels in patient ascites-derived tumor cells maintained under anchorage independence±rhBMP9 for 48 hours and normalized to untreated in accordance with embodiments of the present disclosure.

FIG. 2J shows a Western blot for SOX2 following treatment with rhBMP2 or rhBMP9 for 24 hours or control in cell lines of different cancer origin in accordance with embodiments of the present disclosure.

FIG. 2K shows a qRT-PCR analysis of relative SOX2 increases in anchorage-independent conditions (3D) compared with attached (2D) culture conditions after 72 hours in accordance with embodiments of the present disclosure.

FIG. 2L is a qRT-PCR analysis of relative SOX2±rhBMP2 or rhBMP9 for 24 hours in a 72-h period under anchorage-independent (3D) conditions or attached (2D) conditions in accordance with embodiments of the present disclosure.

FIG. 3A is a Western blot of SOX2 protein following rhBMP9 treatment with indicated doses for 24 h in PA1 and OVCAR3 cells in accordance with embodiments of the present disclosure.

FIGS. 3B-C show a time course analysis of SOX2 protein by western blot (top) and relative SOX2 mRNA by qRT-PCR analysis (bottom) after 10 nM rhBMP2 and rhBMP9 treatment, normalized to untreated conditions in (B) PA1 and (C) OVCAR3 cells in accordance with embodiments of the present disclosure.

FIG. 3D shows a pGL3-SOX2 promoter-reporter luciferase analysis in HEK293 cells following rhBMP2 and rhBMP9 treatment for 24 hours and normalized to untreated and renilla internal control in accordance with embodiments of the present disclosure.

FIGS. 3E-F show Western blot analysis of the effect of rhBMP2 and rhBMP9 treatment for 24 h on SOX2 expression in CMV-CTL and CMV-SOX2 cells in (E) SKOV3 and EF1a-CTL and EF1a-SOX2 in (F) PA1 cells in accordance with embodiments of the present disclosure.

FIG. 3G shows live-dead images from SKOV3 CMV-CTL and CMV-SOX2 cells cultured under anchorage independence for 72 h (top), quantified relative to CMV-CTL control in accordance with embodiments of the present disclosure.

FIG. 3H shows images from SKOV3 CMV-CTL and CMV-SOX2 cells±equimolar rhBMP2 or rhBMP9 for 24 h and live/dead ratios quantified relative to untreated controls below in accordance with embodiments of the present disclosure.

FIG. 4A shows the concentration of indicated ligands in OC patient-derived ascitic fluid in accordance with embodiments of the present disclosure.

FIG. 4B shows a Western blot of SOX2 after treatment with indicated growth factors in PA1 cells in accordance with embodiments of the present disclosure.

FIG. 4C shows a qRT-PCR of SOX2 after treatment with indicated growth factors for indicated times in PA1 cells in accordance with embodiments of the present disclosure.

FIG. 4D shows a time course analysis of SOX2 by qRT-PCR after TGF-β1 treatment in indicated cells in accordance with embodiments of the present disclosure.

FIG. 4E shows images from live-dead analysis upon TGF-β1 treatment in indicated cells in accordance with embodiments of the present disclosure.

FIG. 4F shows a pGL3-SOX2 promoter-reporter luciferase analysis upon TGF-β1 treatment for 24 h in HEK293 cells normalized to untreated and renilla internal control in accordance with embodiments of the present disclosure.

FIG. 4G shows a Western blot of SOX2 after co-treatment of equimolar (1 nM) TGF-β1 and rhBMP9 for 24 h in PA1 cells in accordance with embodiments of the present disclosure.

FIG. 5A shows a Western blot (top) and qRT-PCR (bottom) of SOX2 in PA1 cells pretreated with 5 μM ALK2,3,6 inhibitor dorsomorphin (DM) and 5 μM ALK4/5/7 inhibitor SB431542 for 1 h, followed by treatment with rhBMP9 for 24 hours in accordance with embodiments of the present disclosure.

FIG. 5B shows a Western blot (top) and qRT-PCR (bottom) analysis of SOX2 in PA1 cells pretreated with 5 μM DM and 5 μM SB431542 for 1 h, followed by treatment with rhBMP2 for 24 h in accordance with embodiments of the present disclosure.

FIG. 5C shows a Western blot (left) and qRT-PCR (right) analysis of SOX2 in PA1 cells pretreated with 3 μM ALK1,2 inhibitor ML347 and 0.8 μM ALK2,3 LDN193189 for 1 hour, followed by treatment with rhBMP2/9 for 24 hours in accordance with embodiments of the present disclosure.

FIGS. 5D-E show a Western blot of SOX2 in cells expressing ALK2QD, ALK3QD, or vector control treated for 24 hours with equimolar rhBMP2 and rhBMP9 in (D) PA1 and (E) OVCAR3 cells in accordance with embodiments of the present disclosure.

FIGS. 5F-G show qRT-PCR of SOX2 in indicated cells pretreated with 5 μM SB431542 for 1 h, followed by treatment with 400 pM TGF-β1 for 24 h in accordance with embodiments of the present disclosure.

FIG. 6A shows a qRT-PCR of SMAD1 levels in shSMAD1 cells normalized to shNTC in OVCAR3 cells (left) and Western blot analysis of SOX2 in OVCAR3 shSMAD1 or non-targeting control (shNTC) cells treated with indicated equimolar rhBMPs for 24 hours (right) in accordance with embodiments of the present disclosure.

FIG. 6B shows a qRT-PCR analysis of SMAD3 in OVCAR3 cells transiently expressing siRNA to SMAD3 (siSMAD3) or scramble control (siScr) in accordance with embodiments of the present disclosure.

FIG. 6C shows an in silico analysis showing primers flanking SMAD1 and SMAD3-binding elements (BE) in chromosomal regions in accordance with embodiments of the present disclosure.

FIG. 6(D) Relative qRT-PCR of indicated regions (primer sites) after chromatin immunoprecipitation (ChIP) of SMAD1 with or without 1 h of rhBMP9 treatment, expressed as the ratio over IgG controls normalized to untreated cells (n=2 biological replicates).

FIG. 6E shows a qRT-PCR of indicated regions (primer sites) after ChIP of SMAD3 with or without 1 h of TGF-β1 treatment, expressed as the ratio over IgG controls normalized to untreated cells in accordance with embodiments of the present disclosure.

FIG. 6F shows a qRT-PCR of indicated regions (primer sites) associated with H3K27me3 enrichment with and without 1 hour of rhBMP9 treatment±LDN193189 as indicated in PA1 cells expressed as the ratio over IgG controls normalized to untreated cells in accordance with embodiments of the present disclosure.

FIG. 6G shows a qRT-PCR analysis of SOX2 levels in indicated cells pretreated with 5 μM GSK126 for 5 days, followed by treatment with rhBMP9 for 24 h. Data are normalized to DMSO controls in accordance with embodiments of the present disclosure.

FIG. 6H shows a qRT-PCR of indicated regions (primer sites) after ChIP with H3K4me3±1-h TGF-β1±SB431542 as indicated in PA1 cells expressed as the ratio over IgG controls normalized to untreated cells in accordance with embodiments of the present disclosure.

FIG. 6I shows MS-qPCR using primers proximal to SOX2's TSS (MSP in C) in PA1 cells pretreated with 5 μM 5′-azacytidine (5′-Aza) for 48 h, followed by treatment with rhBMP9 for 24 h, normalized to DMSO control in accordance with embodiments of the present disclosure.

FIGS. 6J-K show qRT-PCR analysis of SOX2 in (J) PA1 and (K) SKOV3 cells treated with 5 μM 5′-Aza and 10 nM rhBMP9, normalized to DMSO control in accordance with embodiments of the present disclosure.

FIG. 7A shows a confocal image from siNTC or siSOX2 PA1 cells under anchorage independence for 72 h (left) with quantitation of live/dead ratio (bottom right), in accordance with embodiments of the present disclosure.

FIG. 7B shows images from PA1 shPLKO.1 and shSOX2 cells under anchorage independence for 72 hours (left). Western blot of SOX2 in shPLK0.1 and shSOX2 cells (top right), and quantitation of live/dead ratio in spheroid cells in accordance with embodiments of the present disclosure.

FIG. 7C shows a Volcano plot of significant DEGs based on adjusted p value of 0.05 between siNTC and siSOX2 in PA1 cells under anchorage independence for 48 hours in accordance with embodiments of the present disclosure.

FIG. 7D shows a Venn diagram of common DEGs between RNA-seq data from (A) and microarray data from rhBMP9 treatment under anchorage independence in PA1 cells from FIG. 2A in accordance with embodiments of the present disclosure . . .

FIGS. 7E and F show gene set enrichment analysis (GSEA) of pathways differentially altered in (E) siSOX2 and (F) siNTC with corresponding Blue-Pink O'gram of core enrichment genes generated by GSEA (right) in accordance with embodiments of the present disclosure.

FIGS. 8A-B shows a kinetics and dose course of indicated rhBMP9 concentration (treatment starting from time 0 hours) on cell survival rate under anchorage independence in (A) HEY parental and (B) HEY T30 cells in accordance with embodiments of the present disclosure.

FIGS. 9A-B shows a kinetics and dose course of indicated rhBMP9 concentration (treatment starting from time 0 h) on cell survival rate under anchorage independence in (A) A2780ip2 and (B) A2780CP cells in accordance with embodiments of the present disclosure.

FIG. 10A shows cell viability of paclitaxel in HEYT30 simultaneously treated with indicated doses of rhBMP9 for 48 hours in accordance with embodiments of the present disclosure.

FIG. 10B shows cell viability of paclitaxel in HEYT30 cells with 24 hr prior rhBMP9 stimulation, followed by combination treatment of rhBMP9 and paclitaxel for 48 hours in accordance with embodiments of the present disclosure.

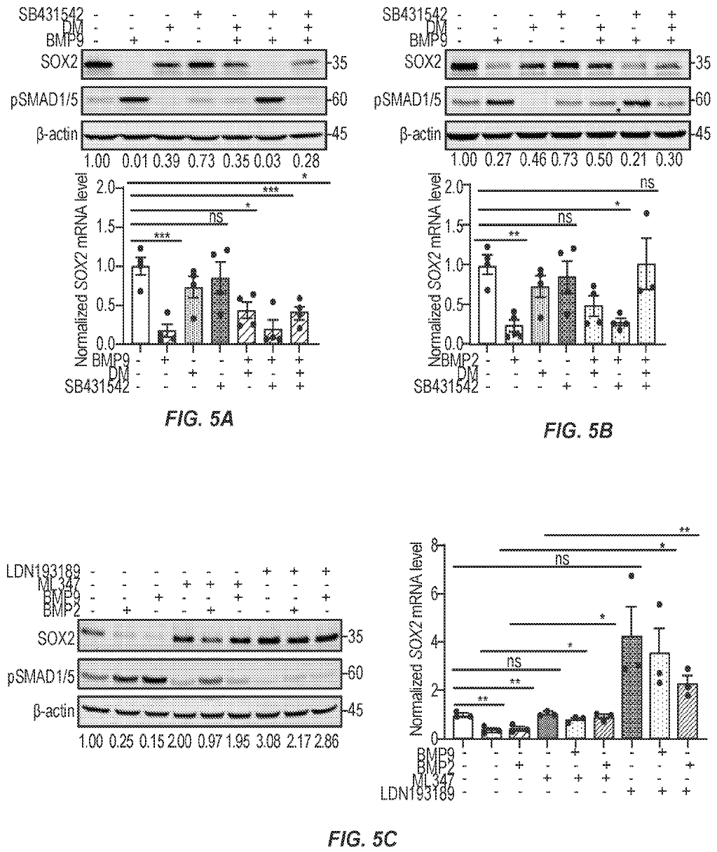

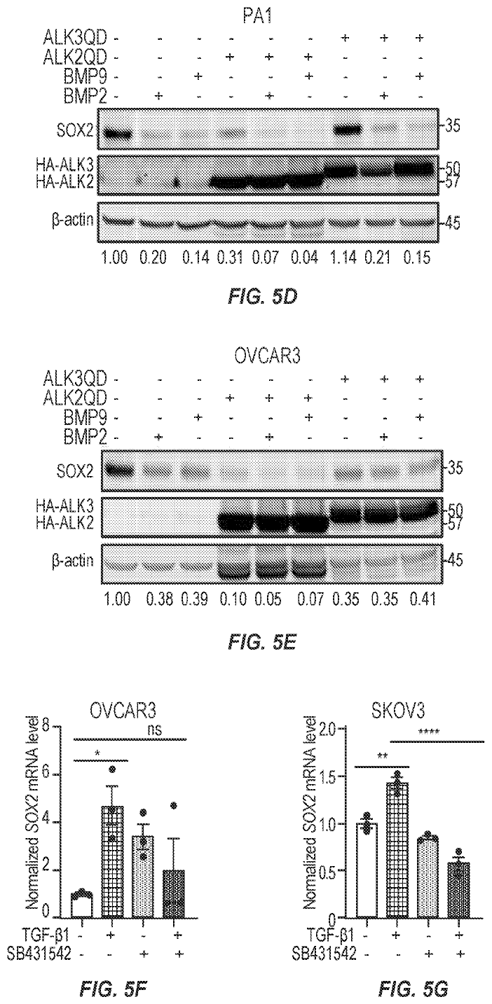

FIG. 5C shows quantification of IC50 of n=2 biological trials for both FIGS. 13A and B in accordance with embodiments of the present disclosure.

FIG. 11 illustrates cell viability of paclitaxel in HEYT30 treated 24 hours prior with rhBMP9, followed by combination treatment of rhBMP9 and paclitaxel for 48 hours in accordance with embodiments of the present disclosure.

FIG. 12 shows a clonogenic assay in HEYT30 cells treated with 80 nM paclitaxel, 10 nM rhBMP9 or combination for 7 days in accordance with embodiments of the present disclosure.

FIGS. 13A-B show co-treatment with indicated dose of rhBMP and paclitaxel in HEY-T30 cells with 24 hours prior 10 nM rhBMP9 stimulation, followed by combination treatment of BMP9 and paclitaxel for 48 hours under 2D (A) and 3D (B) conditions in accordance with embodiments of the present disclosure.

FIG. 13C shows a pictorial representation of HEY-T30 Cell treated with indicated dose of paclitaxel with 24 hours prior 10 nM rhBMP9 stimulation, followed by combination treatment of rhBMP9 and paclitaxel for 48 hours in accordance with embodiments of the present disclosure.

FIGS. 14 shows flow cytometry analysis in HEY-T30 cells treated with 10 nM rhBMP9, 300 nM paclitaxel or combination under 3D conditions for 48 hours, followed by staining with PI and annexin-V in accordance with embodiments of the present disclosure.

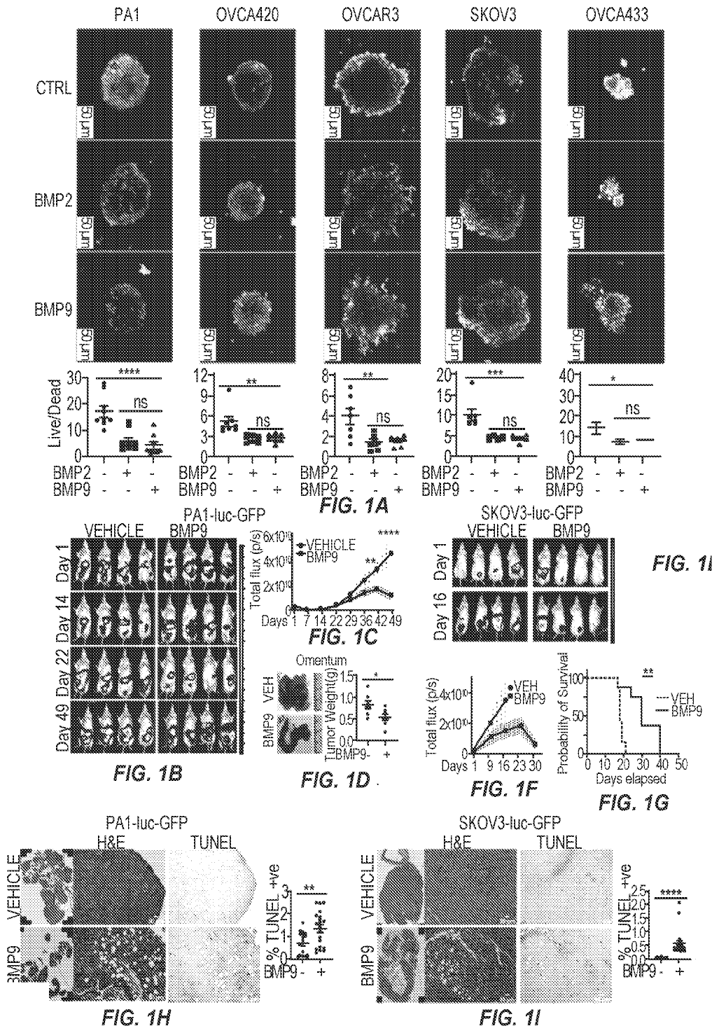

FIG. 15A shows percent dead cells in OVCA cell lines from FIG. 1A cultured under anchorage independence for 48 hours, and subsequently treated with either vehicle (VEH) control or with 10 nM rhBMP2 or rhBMP9 for 24 hours in accordance with embodiments of the present disclosure.

FIG. 15B shows 3D matrigel invasion assay of spheroids in the presence of control, rhBMP2 or rhBMP9 (10 nM) (left) in accordance with embodiments of the present disclosure.

FIG. 15C shows growth curve of PA1 cells grown under attached 2D conditions in the presence of control, rhBMP2 or rhBMP9 in accordance with embodiments of the present disclosure.

FIG. 15D shows body weight in grams of NOD-SCID mice receiving either vehicle or BMP9 at indicated doses for a 21 days period in accordance with embodiments of the present disclosure.

FIG. 15E shows Alanine Transaminase (ALT) concentration as a measure of liver function measured from plasma in accordance with embodiments of the present disclosure.

FIG. 15F shows additional representative images showing complete omental infiltration in vehicle receiving mice as compared to rhBMP9 receiving mice injected with PA1-luc-GFP (left) and SKOV3-luc-GFP (right) in accordance with embodiments of the present disclosure.

FIG. 15G shows representative necrotic region (dark brown) in tumors from rhBMP9 vs vehicle receiving mice injected with SKOV3-luc-GFP in accordance with embodiments of the present disclosure.

FIG. 16A shows a heatmap of transcription profile of 48,226 genes in PA1 cells treated with rhBMP9 for 24 hrs under anchorage independence in accordance with embodiments of the present disclosure.

FIG. 16B shows a REACTOME pathway analysis of genes from data in FIG. 16A in accordance with embodiments of the present disclosure.

FIG. 16C shows a qRTPCR analysis of SOX2 after rhBMP treatment for 24 hrs under anchorage independence (3D) in PA1 cells in accordance with embodiments of the present disclosure.

FIG. 16D shows a qRT-PCR analysis of OCT4 and NANOG after rhBMP9 treatment under 3D condition in PA1 cells in accordance with embodiments of the present disclosure.

FIG. 16E shows images of PA1 cells cultured under anchorage independence for 48 hours, and subsequently treated with either vehicle control or with 10 nM rhBMP4 for 24 hrs in accordance with embodiments of the present disclosure.

FIG. 16F shows images of PA1 cells cultured under anchorage independence for 48 hours, and subsequently treated with either vehicle control or with 10 nM rhBMP10 for 24 hrs in accordance with embodiments of the present disclosure.

FIG. 16G shows a qRT-PCR analysis of SOX2 in tumors from vehicle and rhBMP9 treated groups in SKOV3-luc-GFP mice in accordance with embodiments of the present disclosure.

FIG. 17 shows a Western blot (left) and normalized qRT-PCR (right) of SOX2 expression under attached (2D) versus under anchorage independence (3D) conditions in OVCAR3 cells after 72 hrs under 3D condition in accordance with embodiments of the present disclosure.

FIG. 18A shows a relative qRT-PCR analysis of SOX2 after TGF-β1 treatment for 24 hours under anchorage independence (3D) condition in OVCAR3 cells in accordance with embodiments of the present disclosure.

FIG. 18B shows a live-dead analysis of cells under anchorage independence after (1 nM) TGFβ1 and (10 nM) activin treatment for 24 hrs in indicated cells in accordance with embodiments of the present disclosure.

FIG. 18C shows a pGL3-SOX2 promoter-reporter luciferase analysis upon 10 nM activin A treatment for 24 hours in indicated cells normalized to untreated and renilla internal control in accordance with embodiments of the present disclosure.

FIG. 18D shows a Western blot of SOX2 after combined treatment of equimolar (10 nM) activin and rhBMP2/9 for 24 hrs in PA1 cells in accordance with embodiments of the present disclosure.

FIG. 19A shows a qRT-PCR analysis of SOX2 expression in OVCAR3 cells pretreated with 5 μM Dorsomorphin (DM) and 5 μM SB431542 for 1 hr, followed by treatment with rhBMP9 for 24 hrs in accordance with embodiments of the present disclosure.

FIG. 19B shows a qRT-PCR analysis of SOX2 in OVCAR3 cells pretreated with 5 μM Dorsomorphin (DM) and 5 μM SB431542 for 1 hr, followed by treatment with rhBMP2 for 24 hours in accordance with embodiments of the present disclosure.

FIG. 19C shows a Western blot of SOX2 levels in OVCAR3 cells pretreated with 3 μM ALK1,2 inhibitor ML347 and 0.8 μM ALK2,3 LDN193189 for 1 hr, followed by treatment with rhBMP2/9 for 24 hours in accordance with embodiments of the present disclosure.

FIG. 20 shows a relative qRT-PCR of indicated regions (primer sites) after chromatin immunoprecipitation of SMAD3 to sites on SOX2 proximal chromosomal regions with or without 1 hr of activin A treatment, as indicated in PA1 cells expressed as the ratio over IgG controls normalized to untreated cells in accordance with embodiments of the present disclosure.

FIG. 21A shows a Western blot of SOX2 levels in PA1 cells pretreated for 1 hour with 0.5 μM MG132, followed by rhBMP2/9 in accordance with embodiments of the present disclosure.

FIG. 21B shows a time and dose course of the effect of GSK126 on H3K27me3 levels in indicated cell lines in accordance with embodiments of the present disclosure.

FIG. 21C shows a qRT-PCR analysis of SOX2 expression in cells treated with 10 nM rhBMP9 and 5 μM GSK 126 for 7 days in OVCAR3 in accordance with embodiments of the present disclosure.

FIG. 21D shows a MS-qPCR analysis of SOX2 in SKOV3 cells pretreated with or without 5 μM 5′-Aza for 48 hours, normalized to DMSO control in accordance with embodiments of the present disclosure.

FIG. 21E shows a qRT-PCR analysis of SOX2 in SKOV3 cells treated with or without 5 μM 5′-Aza, normalized to DMSO control in accordance with embodiments of the present disclosure.

FIG. 22 shows a transcriptomic analysis of SOX2 silencing during anchorage independent survival (suspension cultures) in accordance with embodiments of the present disclosure.

### DETAILED DESCRIPTION

The subject matter of embodiments of the present invention is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

### Introduction

Described herein is a method for reducing metastasis of cancer in a subject with cancer by administering an effective amount of BMP9 or an agonist thereof to the subject with cancer. Without being bound by theory, it is believed that by administering BMP9 or an agonist thereof, metastasis of cancer may be reduced because BMP9 strongly enhanced anoikis and suppresses metastasis.

As used throughout, cancer refers to any cellular disorder in which the cells proliferate more rapidly than normal tissue growth. A proliferative disorder includes, but is not limited to, neoplasms, which are also referred to as tumors. A neoplasm can include, but is not limited to, pancreatic cancer, breast cancer, brain cancer (e.g., glioblastoma), lung cancer, a central nervous system cancer, prostate cancer, colorectal cancer, head and neck cancer, ovarian and related gynecological cancer, thyroid cancer, renal cancer, bladder cancer, adrenal cancer and liver cancer A neoplasm can be a solid neoplasm (e.g., sarcoma or carcinoma) or a cancerous growth affecting the hematopoietic system. In some examples, the cancer is a triple negative (estrogen receptors negative (ER−), progesterone receptors negative (PR−) and HER2 negative (HER2−)) breast cancer. Examples of hematopoietic malignanacies include, but are not limited to, myelomas, leukemias, lymphomas (Hodgkin's and non-Hodgkin's forms), T-cell malignancies, B-cell malignancies, and lymphosarcomas. Here, while gynecological cancers are the primary focus, the cancer to be treated is not limited thereto. In some aspects, the cancer is selected from the group consisting of breast cancer, lung cancer, gynecological cancer, neuroendocrine cancer, and combinations thereof. In some aspects, the cancer is ovarian cancer.

The BMP9 may be recombinant BMP9, also referred to as rBMP9. The BMP9 may also be a BMP9 agonist, including a receptor agonist, a signaling pathway agonist, and combinations of BMP9 and an agonist thereof.

### Treatment

In the methods provided herein, a reduction in metastasis refers to the reduction is the size of tumors, slowing of the spread of cancer cells into the peritoneal cavity, organs including liver, omentum, peritoneum, intestinal lining and/or into lymph nodes and via circulation to lungs and bones. Particularly for ovarian cancer, the initial steps of metastasis are regulated by a controlled interaction of adhesion receptors and proteases, and metastasis is characterized by tumor nodules on mesothelium covered surfaces, causing ascites, bowel obstruction, and tumor cachexia.

The term “treatment”, as used herein, refers to any type of therapy, which aims at terminating, preventing, ameliorating or reducing the susceptibility to a clinical condition as described herein. In a preferred embodiment, the term treatment relates to prophylactic treatment (i.e., a therapy to reduce the susceptibility to a clinical condition), of a disorder or a condition as defined herein. Thus, “treatment,” “treating,” and their equivalent terms refer to obtaining a desired pharmacologic or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse effect attributable to the disorder. That is, “treatment” includes (1) preventing the disorder from occurring or recurring in a subject, (2) inhibiting the disorder, such as arresting its development, (3) stopping or terminating the disorder or at least symptoms associated therewith, so that the host no longer suffers from the disorder or its symptoms, such as causing regression of the disorder or its symptoms, for example, by restoring or repairing a lost, missing or defective function, or stimulating an inefficient process, or (4) relieving, alleviating, or ameliorating the disorder, or symptoms associated therewith, where ameliorating is used in a broad sense to refer to at least a reduction in the magnitude of a parameter, such as inflammation, pain, or immune deficiency to a reduction in growth and symptoms an increase in the responsiveness of the cancer to treatment. As described herein, it is envisaged that the BMP9 or an agonist thereof may be administered before, after, or with an anti-cancer compound to treat the cancer.

Any of the methods provided herein can further comprise administering an anti-cancer compound, e.g., a chemotherapeutic agent, prior to, concurrently or after administration of the BMP9 to the subject. Examples of anti-cancer compounds include, but are not limited to avastin, adriamycin, dactinomycin, bleomycin, vinblastine, acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, daunorubicin hydrochloride, decitabine, dexormaplatin, dezaguanine, dezaguanine mesylate, diaziquone, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, interleukin II (including recombinant interleukin II, or rIL2), interferon alfa-2a, interferon alfa-2b, interferon alfa-nl, interferon alfa-n3, interferon beta-1a, interferon gamma-1 b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride.

Any of the methods provided herein can optionally further include administering radiation therapy to the subject. Any of the methods provided herein can optionally further include surgery.

As used throughout, by subject is meant an individual. Preferably, the subject is a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

Throughout, treat, treating, and treatment refer to a method of reducing or delaying one or more effects or symptoms of cancer or metastasis. In some examples, the cancer is ovarian cancer. Treatment can also refer to a method of reducing the underlying pathology rather than just the symptoms. The effect of the administration to the subject can have the effect of, but is not limited to, reducing one or more symptoms (e.g., reduced pain, reduced size of the tumor, etc.) of the cancer, an increase in survival time, a decrease or delay in metastasis, enhancing T cell function (e.g., proliferation, cytokine production, tumor cell killing), a reduction in the severity of the cancer (e.g., reduced rate of growth of a tumor or rate of metastasis), increasing latency between symptomatic episodes, decreasing the number or frequency of relapse episodes, the complete ablation of the cancer or a delay in the onset or worsening of one or more symptoms. For example, a disclosed method is considered to be a treatment if there is about a 10% reduction in one or more symptoms of the disease in a subject when compared to the subject prior to treatment or when compared to a control subject or control value. Thus, the reduction can be about a 10, 20, 30, 40, 50, 60, 70, 80, 90, 100%, or any amount of reduction in between.

As used throughout by prevent, preventing, or prevention is meant a method of precluding, delaying, averting, obviating, forestalling, stopping, or hindering the onset, incidence, severity, or recurrence of cancer, such as ovarian cancer. For example, the disclosed method is considered to be a prevention if there is a reduction or delay in onset, incidence, severity, or recurrence of cancer, such as gynecological cancer cancer or one or more symptoms of cancer, such as ovarian cancer (e.g., relapse, disease progression, increase in tumor size, metastasis) in a subject treated with a BMP9 or an agonist thereof and a cisplatin or paclitaxel therapy as compared to control subjects treated with cisplatin or paclitaxel therapy that did not receive BMP9 or an agonist thereof. The disclosed method is also considered to be a prevention if there is a reduction or delay in onset, incidence, severity, or recurrence of cancer, such as ovarian cancer or one or more symptoms of cancer, such as ovarian cancer in a subject after receiving BMP9 as compared to the subject's progression prior to receiving treatment. Thus, the reduction or delay in onset, incidence, severity, or recurrence of a cancer, such as ovarian cancer, can be about a 10, 20, 30, 40, 50, 60, 70, 80, 90, 100%, or any amount of reduction in between.

A biological sample can be any sample obtained from an organism. Examples of biological samples include body fluids and tissue specimens. For example, the sample can be a tissue biopsy, for example, a tumor biopsy. The source of the sample may also be physiological media such as blood, serum, plasma, cerebral spinal fluid, breast milk, pus, tissue scrapings, washings, urine, feces, tissue, such as lymph nodes, spleen, ascites fluid, peritoneal washings or the like. The term tissue refers to any tissue of the body, including blood, connective tissue, epithelium, contractile tissue, neural tissue, and the like.

In the methods provided herein, methods standard in the art for quantitating nucleic acids may be used and are described in detail further herein. These include, but are not limited to, in situ hybridization, quantitative PCR, RT-PCR, Taqman assay, Northern blotting, ELISPOT, dot blotting, etc., as well as any other method now known or later developed for quantitating the amount of a nucleic acid in a cell or released from a cell. Additional methods standard in the art for quantitating proteins such as densitometry, absorbance assays, fluorometric assays, Western blotting, ELISA, radioimmunoassay, ELISPOT, immunoprecipitation, immunofluorescence (e.g., FACS), immunohistochemistry, etc., as well as any other method now known or later developed for quantitating a specific protein in or produced by cells in a sample may be used.

The term effective amount, as used throughout, is defined as any amount of an agent (for example, BMP9, a chemotherapeutic agent, etc.) necessary to produce a desired physiologic response. Exemplary dosage amounts for a mammal include doses from about 0.5 to about 200 mg/kg of body weight of active compound per day, which may be administered in a single dose or in the form of individual divided doses, such as from 1 to 4 times per day can be used. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 0.5 to about 15 mg/kg of body weight of active compound per day, about 0.5 to about 10 mg/kg of body weight of active compound per day, about 0.5 to about 5 mg/kg of body weight of active compound per day, about 1 to about 20 mg/kg of body weight of active compound per day, about 1 to about 10 mg/kg of body weight of active compound per day, about 1 to about 5 mg/kg of body weight of active compound per day, about 20 mg/kg of body weight of active compound per day, about 10 mg/kg of body weight of active compound per day, or about 5 mg/kg of body weight of active compound per day. One of skill in the art would adjust the dosage as described below based on specific characteristics of the agent and the subject receiving it.

Effective amounts and schedules for administering the agent can be determined empirically and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, unwanted cell death, and the like. Generally, the dosage will vary with the type of inhibitor, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

In some aspects, the BMP9, e.g., rBMP9, or an agonist thereof may be administered in an amount ranging from 0.01 mg/kg to 50 mg/kg, e.g., 0.01 mg/kg, 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg.kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.7 mg/kg, 4.8 mg/kg, 4.9 mg/kg, 5.0 mg/kg, 6.0 mg/kg, 10 mg/kg, 15 mg/kg, 20 mg/kg, 25 mg/kg, 30 mg/kg, 35 mg/kg, 40 mg/kg, 45 mg/kg, 50 mg/kg, or and combination of values and ranges disclosed herein.

In some aspects, the BMP9, e.g., rBMP9, or an agonist thereof, may be administered daily for a certain period of time, as such a period of time before or alongside an anti-cancer compound is to be administered. For example, the BMP9 may be administered for a period of at least 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or even longer, such as for up to 60 days, 90 days, 120 days, 180 days, or longer. The administration may be once a day, twice a day, or more frequently. There may also be a break between administration, such as a break of last least 12 hours, at least 24 hours, at least 36 hours, at least 48 hours, at least 72 hours, at least 96 hours, at least 120 hours, at least 144 hours, at least one week, at least one week, at least one month, at least 3 months, or at least 6 months. These values may also be used as upper limits for the time between administration.

Any of the agents described herein can be provided in a pharmaceutical composition. These include, for example, a pharmaceutical composition comprising a therapeutically effective amount of one or more agents and a pharmaceutically acceptable carrier.

Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22nd edition, Loyd V. Allen et al, editors, Pharmaceutical Press (2012).

Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, New Jersey), polyethylene glycol (PEG), and PLURONICS™ (BASF; Florham Park, NJ).

Compositions containing one or more of the agent(s) described herein suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof are admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

The compositions are administered in any of a number of ways depending on whether local or systemic treatment is desired and on the area to be treated. Any of the compositions described herein can be delivered by any of a variety of routes including by injection (e.g., subcutaneous, intramuscular, intravenous, intra-arterial, intraperitoneal), by continuous intravenous infusion, cutaneously, dermally, transdermally, orally (e.g., tablet, pill, liquid medicine, edible film strip), by implanted osmotic pumps, by suppository, or by aerosol spray. Routes of administration include, but are not limited to, topical, intradermal, intrathecal, intralesional, intratumoral, intrabladder, intravaginal, intra-ocular, intrarectal, intrapulmonary, intracranial, intraventricular, intraspinal, dermal, subdermal, intra-articular, placement within cavities of the body, nasal inhalation, pulmonary inhalation, impression into skin, and electroporation.

In an example in which a nucleic acid is employed the nucleic acid can be delivered intracellularly (for example by expression from a nucleic acid vector or by receptor-mediated mechanisms), or by an appropriate nucleic acid expression vector which is administered so that it becomes intracellular, for example by use of a retroviral vector (see U.S. Pat. No. 4,980,286), or by direct injection, or by use of microparticle bombardment (such as a gene gun; Biolistic, Dupont), or coating with lipids or cell-surface receptors or transfecting agents, or by administering it in linkage to a homeobox-like peptide which is known to enter the nucleus (for example Joliot et al., Proc. Natl. Acad. Sci. USA 1991, 88:1864-8). Nucleic acid carriers also include, polyethylene glycol (PEG), PEG-liposomes, branched carriers composed of histidine and lysine (HK polymers), chitosan-thiamine pyrophosphate carriers, surfactants, nanochitosan carriers, and D5W solution. The present disclosure includes all forms of nucleic acid delivery, including naked DNA, plasmid and viral delivery, integrated into the genome or not. Physical transduction techniques can also be used, such as liposome delivery and receptor-mediated and other endocytosis mechanisms (see, for example, Schwartzenberger et al., Blood 87:472-478, 1996) to name a few examples. This invention can be used in conjunction with any of these or other commonly used gene transfer methods.

Effective doses for any of the administration methods described herein can be extrapolated from dose-response curves derived from in vitro or animal model test systems.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### BMP9 and SOX2

Growth factors in tumor environments are regulators of cell survival and metastasis. The inventors have surprisingly and unexpectedly discovered the dichotomy between TGF-β superfamily growth factors BMP and TGF-β/activin and their downstream SMAD effectors. Gene expression profiling uncovers SOX2 as a key contextual signaling node regulated in an opposing manner by BMP2, -4, and -9 and TGF-β and activin A to impact anchorage-independent cell survival. SOX2 is repressed by BMPs, leading to a reduction in intraperitoneal tumor burden and improved survival of tumor-bearing mice. Repression of SOX2 is driven by SMAD1-dependent histone H3K27me3 recruitment and DNA methylation at SOX2′s promoter. Conversely, TGF-β, which is elevated in patient ascites, and activin A can promote SOX2 expression and anchorage-independent survival by SMAD3-dependent histone H3K4me3 recruitment. Our findings identify SOX2 as a contextual and contrastingly regulated node downstream of TGF-β members controlling anchorage-independent survival and metastasis in ovarian cancers.

Cancer metastasis is dependent on the ability of the tumor cells to acquire anoikis resistance, programmed cell death under anchorage independence. Ascites accumulation in the abdomen is associated with diseases of the peritoneal cavity, with 30% related to ovarian cancer (OC). More than 90% of stages III and IV OC patients present malignant ascites, which harbor cancer cell clusters in suspension that contribute to metastasis. Such tumor cells exhibit anchorage-independent survival due to evasion of anchorage-independent cell death mechanisms (termed anoikis) in the ascites environment enriched with growth factors that can contribute to recurrence and therapy resistance,

Thus, defining specific growth factors that promote cell survival in the ascites and conversely defining strategies that disrupt survival and promote anoikis will improve the ability to control recurrence and mortality of advanced OC patients.

The transforming growth factor β (TGF-β) family of cytokines that include TGF-βs, bone-morphogenetic proteins (BMPs/GDFs), activins, inhibins, glial-derived neurotrophic factors, and nodal have crucial roles in development and cancer. Cellular responses are initiated upon ligand binding to the type I and type II serine threonine kinases and type III cell surface TGF-β receptors. The types I and II receptors form ligand-dependent homomeric and heteromeric complexes to phosphorylate intracellular-receptor-regulated SMADs (R-SMADs). R-SMADs complex with the common SMAD4 and accumulate in the nucleus to regulate gene expression. Although members of the TGF-β superfamily share similarities in the signaling events, they differ in their receptor affinities and the complexes formed. The BMP ligands (BMP2, 4, 9/GDF2, and BMP10) bind type I receptors: ALK1 (ACVRL1), ALK2 (ACVR1), ALK3 (BMPR1A), or ALK6 (BMPR1B), which recruit the type II receptor (BMPR2), leading to phosphorylation and nuclear translocation of the SMAD1/5/8-SMAD4 complex. BMP2 and BMP4 share a high degree of sequence identity and effectively bind ALK2, -3, and -6 receptors BMP9 and BMP10 both have high affinity for ALK1. However, only BMP9 can also interact with ALK2 and ALK3/6 receptors.

TGF-βs and activin, on the other hand, first bind the type II receptor (TβRII/ACTR-IIA/B), which complexes with the type I receptors ALK4 (ACVR1B), ALK5 (TGFβR1), and ALK7 (ACVRIC) to mediate downstream signaling via SMAD2/3. BMPs can also induce SMAD2/3 signaling via ALK3/6 (BMP-binding type I) and ALK5/7 (TGF-β-binding type I) receptors. Similarly, TGF-β1 can also lead to phosphorylation of SMAD1/5 via ALK2/3 and ALK5 receptors. SMAD1/5 activation by activin is seldom seen but has been reported. In addition to cross-utilization of receptors, TGF-β/activin and BMP can both cooperate and antagonize each other. In cancer, both ligands can have tumor-suppressive and-promoting effects However, limited studies have delineated the function and relationship between TGF-β members, including BMPs (2, 4, 9, and 10), TGF-β1, and activin during metastasis. Here, their relationship is delineated in a singular context of OC anchorage-independent survival that impacts metastasis, to elucidate their pathological and signaling relationship. In doing so, SOX2 was identified as a central regulated node downstream of BMP9, BMP2, BMP4, TGF-β1, and activin.

Sex-determining region Y-box 2 (SOX2), is a single-exon transcription factor with key roles in embryonic development and stem cell maintenance. In cancer, SOX2-mediated transcriptional reprogramming is associated with a stem cell fate and tumor-initiating capacity. Although SOX2 is an indicator of premalignant lesions and a proposed biomarker in OC, it has been paradoxically linked to both poor and better outcomes. Thus, defining its precise contextual roles and mechanisms that regulate expression is critical.

Contextual regulation of SOX2 by TGF-β members are demonstrated here. SOX2 is found to be a central repressed target of BMPs (9, 2, 4) leading to suppression of anchorage-independent survival and metastasis. SOX2 repression occurs through DNA and chromatin modification-based mechanisms mediated by SMAD1/5, leading to increased cell death under anchorage independence (referred to as anoikis). Conversely, TGF-β which is significantly elevated in patient ascites, and activin A increase SOX2 expression in a SMAD3-dependent manner, leading to decreased anoikis. Notably, the presence of BMPs and SMAD1 signaling can override the effects of TGF-and activin on SOX2. Our findings implicate the use of a subset of BMPs as a therapeutic strategy and demonstrate a critical role of context-specific SOX2 regulation in controlling anchorage-independent survival and metastasis in ovarian cancer.

An in-depth analysis of TGF-β family members in anchorage-independent survival and OC transcoelomic metastasis by delineating distinct effects of the TGF-β, BMP, and activin subfamilies has previously been published where a role for BMP9 (GDF2) in anoikis was observed. The experimental section below expands the effects of BMP9 to BMP2 and BMP4. BMP9 administration at the time of intraperitoneal tumor cell injection into mice, to mimic the shedding of tumor cell from the primary tumor, reduced transcoelomic metastasis and prolonged overall survival from disease. BMP9 also decreased spheroid invasion, which could be a result of reduced viable cells in the spheroids. The findings on the impact of BMP9 on normalizing tumor blood vessels suggest potential dual effects of the anti-tumor properties of BMP9 on the vasculature likely via the endothelial-specific TGF-β receptor ACVRL1 and an anoikis effect on epithelial cells via ALK2,3, as seen here. Thus, therapeutic strategies that use BMP9 mimetics along with anti-angiogenics may be promising.

SOX2 is at the epicenter, driving the differential effects of BMPs and TGF-β on anoikis. Although both BMP2 and BMP4 also increase anoikis and repress SOX2, BMP9 was more potent. It is possible that BMP2 may involve additional or alternate mediators besides SOX2. BMP10 had limited effects on epithelial cell anoikis, contrasting with BMP9. This may be explained by the ability of BMP9 to utilize ALK2.

Consistent with this receptor specificity hypothesis and model, BMP10 failed to activate SMAD1/5, repress SOX2, and impact anoikis. Probing the receptor mechanisms revealed that ALK2/ALK3-induced phosphorylation of SMAD1 is critical for SOX2 repression. A more robust requirement of ALK2 than ALK3 was observed, which could account for higher sensitivity to BMP9 than BMP2.

Without being bound by theory, it is believed that both BMP2 and BMP9 have been shown to act as tumor suppressors in cancers including, but not limited to, breast and prostate with prior conflicting studies in OC indicating increased tumor growth in subcutaneous models that do not incorporate intraperitoneal cancer spread.

Again, without being bound by theory, it is believed that a key finding here is the divergent role of TGF-β members on anoikis and SOX2. This is of clinical relevance, as patient ascites are highly enriched in TGF-β1 but not in BMP9, suggesting that OC cells are primarily exposed to TGF-β1 that stimulates SOX2 and suppresses anoikis. TGF-β can also regulate epithelial to mesenchymal transition (EMT) via SMAD3, which has been associated with anoikis resistance and spheroid invasion. These findings are consistent with studies on inhibition of TGF-β1 signaling and reduced peritoneal tumor growth in OC.

With accumulating preclinical and clinical evidence on TGF-β1 and activins as a therapeutic target in OC, these findings highlight an important mechanism of their pro-metastatic roles through SOX2 and anoikis resistance.

Since TGF-β1 receptor dependencies are key in the regulation of SOX2 by TGF-β family members, alterations in receptor expression could be an important tipping point in determining the balance between SMAD1 and SMAD3 signaling, leading to SOX2 downregulation or upregulation. TGF-β members, particularly TGF-β1, can also lead to phosphorylation of SMAD1/5 via ALK2/3 and ALK5 receptors. However, SMAD3knockdown was found to be sufficient to abrogate TGF-β1-mediated increases in SOX2. The compensatory increase in pSMAD1 levels that correlated with lowered SOX2 suggest that shifting the balance between SMAD1 activation and SMAD3 activation, regardless of the upstream ALK involved, could potentially tip the effect of exogenous TGF-β from increasing SOX2 to suppressing SOX2.

In ligand combination studies of TGF-β1/activin with BMP9, BMP9 could override TGF-β1/activin to downregulate SOX2. Future in-depth experiments could inform BMP9 therapeutic regimens. In a contrasting but conceptually consistent scenario, high levels of BMP antagonists such as gremlin have been reported in cancer, which might explain the loss of BMP responsiveness and tumor-suppressive function sometimes seen in OC.

The importance of the finding here that SOX2 is a centrally regulated target should be emphasized, given that SOX2 is a pioneer transcription factor and can predict survival and prognosis in multiple cancers

Several epigenetic mechanisms can regulate SOX2 and likely, these are exploited by the SMADs. Both increased SOX2 promoter methylation at the CpG islands and H3K27me3/H3K4me3 enrichment occurred in a SMAD-signaling-dependent manner. H3K27me3 and H3K4me3 enrichment occurred in the same regions as SMAD1 and SMAD3 in response to BMP9 and TGF-β1 respectively, and depended on SMAD activation/phosphorylation. The contribution of DNA methylation in response to BMP9 in conjunction with H3K27me3 is a likely explanation for the high degree of SOX2 repression observed in response to BMP9.

Overall, the inventors determined that both intrinsic cellular states and the growth factor environment strongly influence SOX2, providing information on the effects of changing the balance in growth factors in the ovarian cancer ascites environment, which may inform therapeutic targeting of these pathways in ovarian cancer.

A detailed description of each Figure is included below. The Figures are also discussed in the Examples section.

FIG. 1: BMPs induce anoikis and suppress OC growth and metastasis in vivo.

FIG. 1A shows representative confocal microscopy images of OC cells cultured under anchorage independence for 48 hours and subsequently treated with vehicle (VEH) or 10 nM rhBMP2 or rhBMP9 for 24 hours. Live/dead cell ratios assessed by calcein-AM (green, live cells) and ethidium homodimer dye (red, dead cells). (Scale bar, 50 μm; n=7-10 spheroids/condition).

FIG. 1B shows representative tumor luminescence images of NOD-SCID mice injected with PA1-luc-GFP cells with vehicle or rhBMP9 (5 mg/kg) administered i.p. daily (indicated days post-tumor cell injection from 4 mice are shown).

FIG. 1C shows whole-animal luminescence quantified over time (n=8 mice for rhBMP9, n=7 for vehicle).

FIG. 1D shows representative image of omental tumor burden (left) and quantification of omental tumor weight (right) from mice that received vehicle or rhBMP9 injected with PA1-luc-GFP tumor cells (n=8 mice for rhBMP9, n=7 for vehicle).

FIG. 1E shows representative tumor luminescence images of NOD-SCID mice injected with SKOV3-luc-GFP cells with vehicle or rhBMP9 (5 mg/kg) administered i.p. daily (days 1 and 16 post-tumor cell injection from 4 mice are shown).

FIG. 1F shows whole-animal luminescence quantified over time (n=8 for rhBMP9, n=7 for vehicle).

FIG. 1G a KM plot of SKOV3-Luc-GFP-injected mice receiving rhBMP9 compared with vehicle.

FIG. 1H shows representative H&E and TUNEL staining of PA1-luc-GFP tumors, and FIG. 1I SKOV3-luc-GFP tumors. (Scale bar, 50 mm; TUNEL stain quantification is shown for two mice per group per cell line. n=17-20 random fields/condition) (right: *p<0.05, **p<0.01, ***p<0.001). All data are the mean±SEM; *p<0.05, **p<0.01, ***p<0.001. Statistical significance determined by (A) ANOVA followed by Dunnett's multiple comparison test and (C-I) unpaired t test (see also FIG. 15).

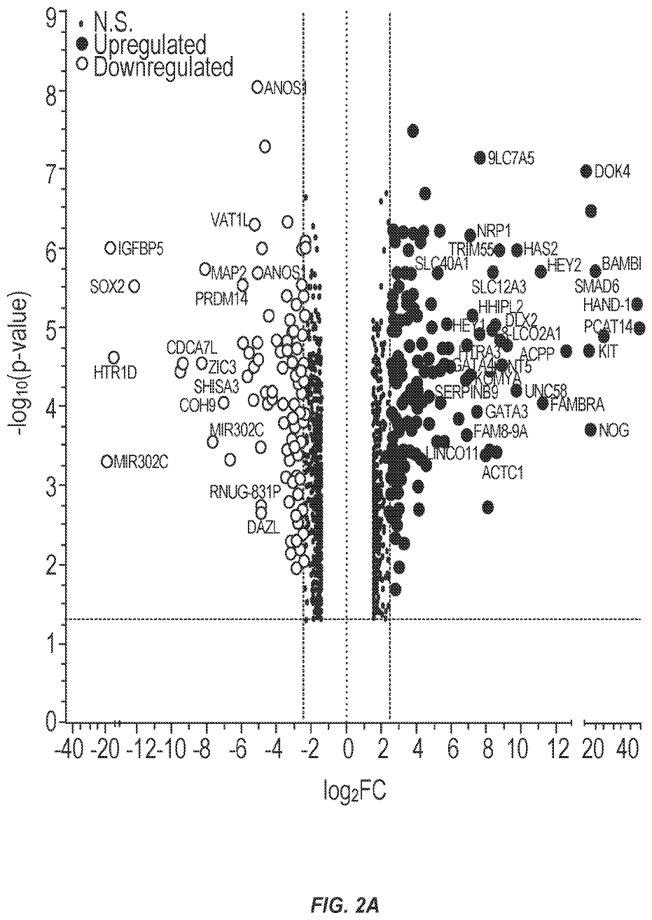

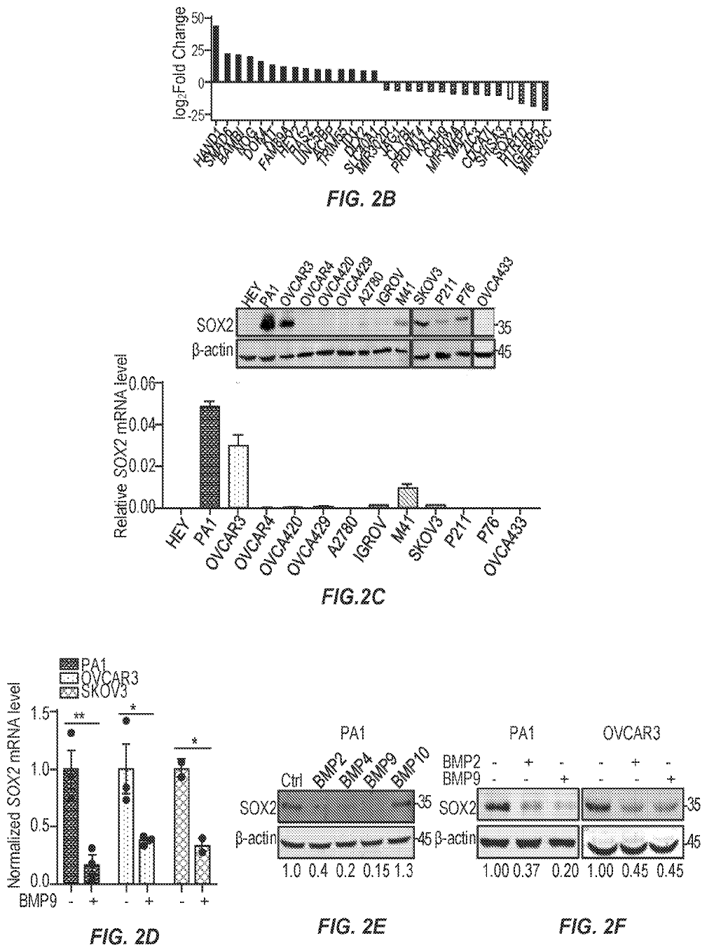

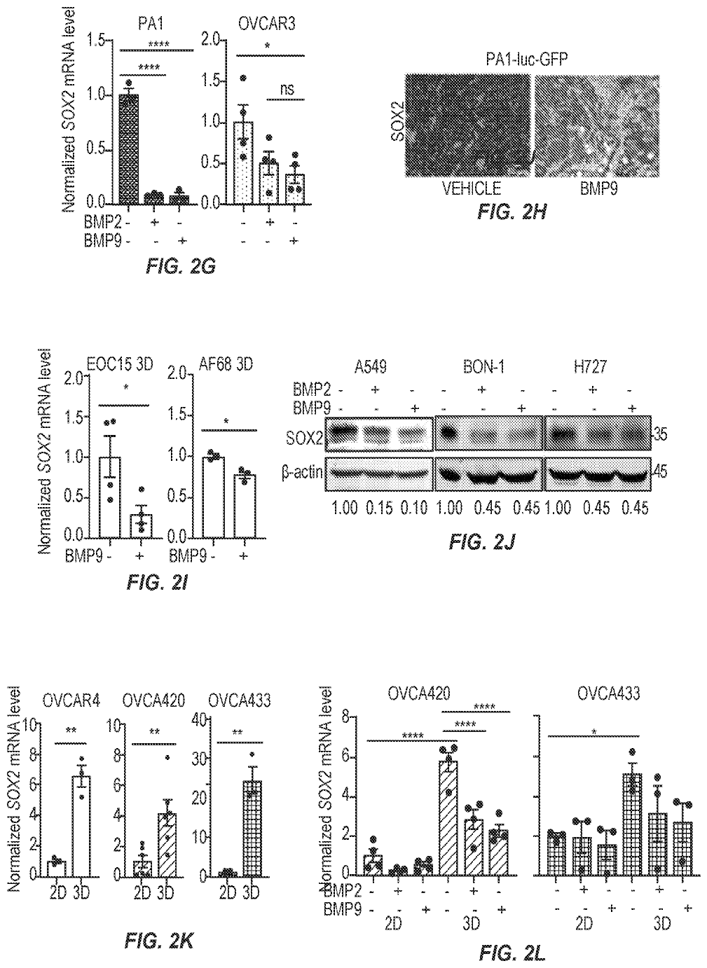

FIG. 2: SOX2 is downregulated by BMP2, -4, and -9 in cancer cell lines and xenograft tumors.

FIG. 2A shows volcano plot of changes in gene expression in PA1 cells under anchorage-independence treated with vehicle or rhBMP9 for 24 h (cutoff 1.5 log₂ FC; genes above cutoff of 5 are labeled; n=3 biological replicates).

FIG. 2B list of 15 top upregulated and downregulated genes in response to rhBMP9 from 2A.

FIG. 2C shows Western blot (WB; top) and qRT-PCR (bottom) of SOX2 levels in a panel of OC cells (WB: n=2-5 biological replicates; qPCR: n=3 technical replicates).

FIG. 2D shows qRT-PCR analysis of SOX2 mRNA levels in response to 24 hours of rhBMP9 (10 nM) treatment expressed relative to control untreated cells (n=2-3 biological replicates).

FIG. 2E shows Western blot following treatment with rhBMP2, -4, and -9 and 10 (10 nM) or control for 24 hours in PA1 cells to assess SOX2 protein (n=2-3 biological replicates). Quantitation of SOX2 relative to actin is presented below.

FIG. 2F shows Western blot of rhBMP2 and rhBMP9 treatment and SOX2 protein in PA1 and OVCAR3 cells (n=2 biological replicates). Quantitation of SOX2 relative to actin is presented below.

FIG. 2G shows qRT-PCR analysis of relative SOX2 levels after rhBMP2 and rhBMP9 treatment normalized to untreated control (n=3-4 biological replicate).

FIG. 2H shows representative immunohistochemistry (IHC) of SOX2 in PA1-Luc-GFP tumors from mice receiving vehicle or rhBMP9 from FIG. 1B (Scale bar, 50 μm; n=2 mice/condition).

FIG. 21 shows qRT-PCR analysis of relative SOX2 transcript levels in patient ascites-derived tumor cells maintained under anchorage independence±rhBMP9 for 48 hours and normalized to untreated (n=3-4 biological replicates).

FIG. 2J shows Western blot for SOX2 following treatment with rhBMP2 or rhBMP9 for 24 hours or control in cell lines of different cancer origin (n=2, A459 n=3 biological replicates). Quantitation of SOX2 relative to actin is shown.

FIG. 2K shows qRT-PCR analysis of relative SOX2 increases in anchorage-independent conditions (3D) compared with attached (2D) culture conditions after 72 hours (n=3-6 biological replicates).

FIG. 2L shows qRT-PCR analysis of relative SOX2±rhBMP2 or rhBMP9 for 24 hours in a 72-h period under anchorage-independent (3D) conditions or attached (2D) conditions (n=3-4 biological replicates). Data are normalized to untreated attached (2D) conditions in indicated cells for (K) and (L). Data are the mean±SEM; *p<0.05, **p<0.01, ***p<0.001. Statistical significance determined by ANOVA followed by (D) Sidak's or (G-L) Dunnett's multiple comparison test (see also Figures S2 and S3).

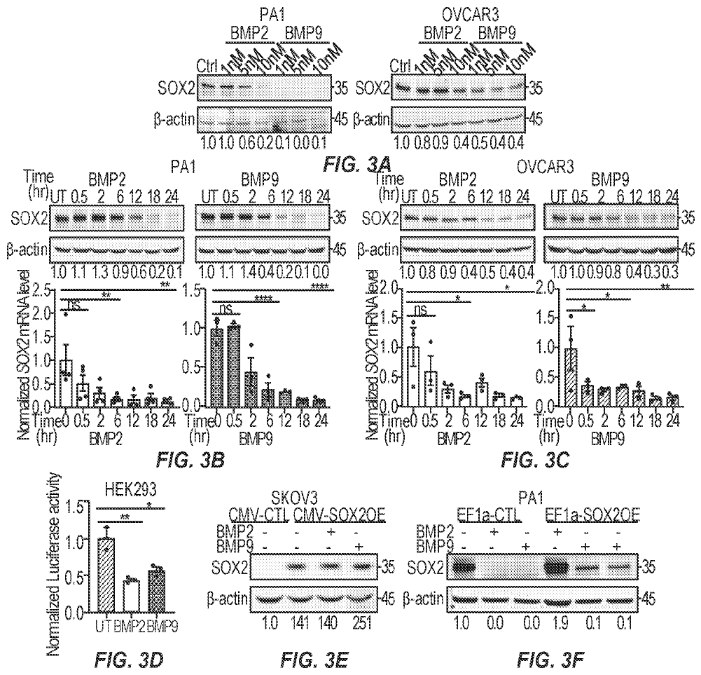

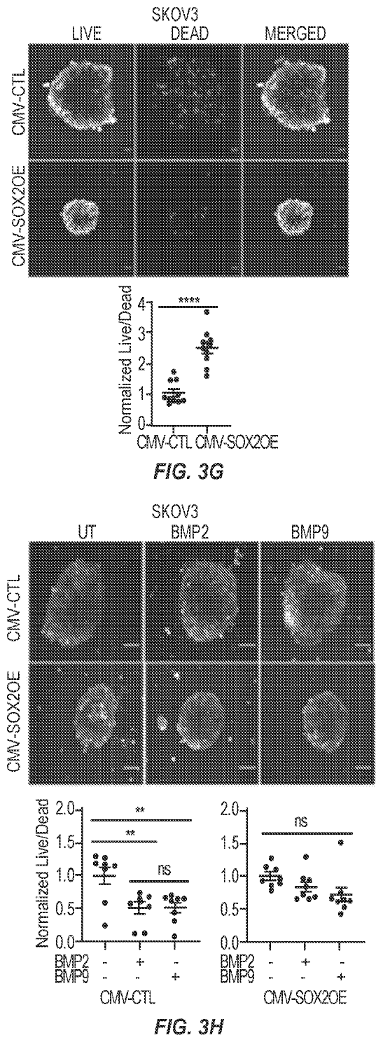

FIG. 3: Downregulation of SOX2 is required for anoikis.

FIG. 3A shows Western blot of SOX2 protein following rhBMP treatment with indicated doses for 24 h in PA1 and OVCAR3 cells. Quantitation of SOX2 relative to actin is presented below (n=3 biological replicates).

FIG. 3B and FIG. 3C show time course analysis of SOX2 protein by western blot (top) and relative SOX2 mRNA by qRT-PCR analysis (bottom) after 10 nM rhBMP2 and rhBMP9 treatment, normalized to untreated conditions (time 0 h/UT) in (B) PA1 and (C) OVCAR3 cells (n=3-4 biological replicates).

FIG. 3D shows pGL3-SOX2 promoter-reporter luciferase analysis in HEK293 cells following rhBMP2 and rhBMP9 treatment for 24 hours and normalized to untreated and renilla internal control (n=2-3 biological replicates).

FIG. 3E and FIG. 3F show Western blot analysis of effect of rhBMP2 and rhBMP9 treatment for 24 h on SOX2 expression in CMV-CTL and CMV-SOX2 cells in (E) SKOV3 (n=3 biological replicates), and EF1a-CTL and EF1a-SOX2 in (F) PA1 cells (n=1 biological replicate).

FIG. 3G shows representative live-dead images from SKOV3 CMV-CTL and CMV-SOX2 cells cultured under anchorage independence for 72 h (top), quantified relative to CMV-CTL control (bottom right, n=10 spheroids/condition; scale bar, 50 μm. unpaired t test).

FIG. 3H shows representative images from SKOV3 CMV-CTL and CMV-SOX2 cells±equimolar rhBMP2 or rhBMP9 for 24 h and live/dead ratios quantified relative to untreated controls below (n=8 spheroids/condition; scale bar, 50 μm). Data are the mean±SEM; *p<0.05, **p<0.01, ***p<0.001. Statistical significance determined by ANOVA followed by Dunnett's multiple comparison test.

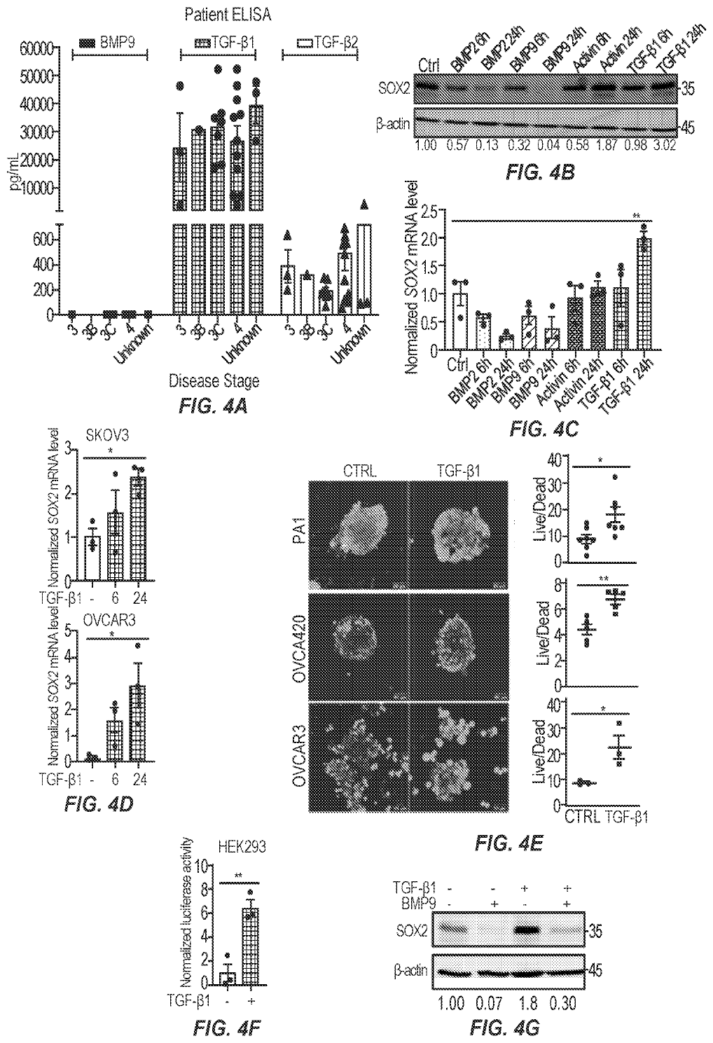

FIG. 4: Ovarian cancer (OC) ascites are high in TGF-β ligands, which upregulate SOX2 transcription and suppress anoikis.

FIG. 4A shows concentration of indicated ligands in OC patient-derived ascitic fluid (rhBMP9, n=10; TGF-β1, n=25; TGFβ2, n=25, where n represents number of patients).

FIG. 4B shows Western blot of SOX2 after treatment with indicated growth factors in PA1 cells. Quantitation of SOX2 relative to actin is presented below (n=3 biological replicates).

FIG. 4C shows qRT-PCR of SOX2 after treatment with indicated growth factors for indicated times in PA1 cells (n=3 biological replicates; ANOVA followed by Dunnett's multiple comparisons test).

FIG. 4D shows time course analysis of SOX2 by qRT-PCR after TGF-β1 treatment in indicated cells (n=3 biological replicates; ANOVA followed by Sidak's multiple comparison test and unpaired Student's t test).

FIG. 4E shows representative images from live-dead analysis upon TGF-β1 treatment in indicated cells. Quantitation of live/dead ratio in spheroids assessed by calcein-AM (green, live cells) and ethidium homodimer dye (red, dead cells; n=3-7 spheroids/condition; scale bar, 50 μm).

FIG. 4F shows pGL3-SOX2 promoter-reporter luciferase analysis upon TGF-β1 treatment for 24 h in HEK293 cells normalized to untreated and renilla internal control (n=3 biological replicates) (unpaired t test).

FIG. 4G shows Representative western blot of SOX2 after co-treatment of equimolar (1 nM) TGF-β1 and rhBMP9 for 24 h in PA1 cells (n=3 biological replicates). Data are the mean±SEM; *p<0.05, **p<0.01, ***p<0.001 (see also FIG. 18).

FIG. 5: SOX2 is reciprocally regulated by ALK2/ALK3 and ALK5 receptor kinases.

FIG. 5A shows Western blot (top) and qRT-PCR (bottom) of SOX2 in PA1 cells pretreated with 5 μM ALK2,3,6 inhibitor dorsomorphin (DM) and 5 μM ALK4/5/7 inhibitor SB431542 for 1 h, followed by treatment with rhBMP9 for 24 hours. Data are normalized to vehicle DMSO controls. Quantitation of SOX2 relative to actin is presented below (WB, n=3 for DM and n=2 biological replicates for SB; qPCR: n=4 biological replicates).

FIG. 5B shows Western blot (top) and qRT-PCR (bottom) analysis of SOX2 in PA1 cells pretreated with 5 μM DM and 5 μM SB431542 for 1 h, followed by treatment with BMP2 for 24 h. Data are normalized to DMSO vehicle controls. Quantitation of SOX2 relative to actin is presented below (n=4 for DM and n=2 biological replicates for SB; qPCR: n=3-4 biological replicates).

FIG. 5C shows Western blot (left) and qRT-PCR (right) analysis of SOX2 in PA1 cells pretreated with 3 μM ALK1,2 inhibitor ML347 and 0.8 μM ALK2,3 LDN193189 for 1 hour, followed by treatment with BMP2/9 for 24 hours. Data are normalized to vehicle controls presented. Quantitation of SOX2 relative to actin is presented below (WB, n=2 biological replicates; qRT-PCR, n=3 biological replicates).

FIG. 5D and FIG. 5E shows Western blot of SOX2 in cells expressing ALK2QD, ALK3QD, or vector control treated for 24 hours with equimolar rhBMP2 and rhBMP9 in (D) PA1 (n=2 biological replicates) and (E) OVCAR3 (n=1 biological replicate) cells.

FIG. 5F and FIG. 5G shows qRT-PCR of SOX2 in indicated cells pretreated with 5 μM SB431542 for 1 h, followed by treatment with 400 pM TGF-β1 for 24 h. Data are normalized to DMSO controls (n=3 biological replicates). All data are presented as the mean±SEM; *p<0.05, **p<0.01, ***p<0.001 (A-B) and (F) ANOVA followed by Dunnett's multiple comparison test, (C) Student t test, and (G) ANOVA followed by Sidak's multiple comparison test (see also FIG. 19).

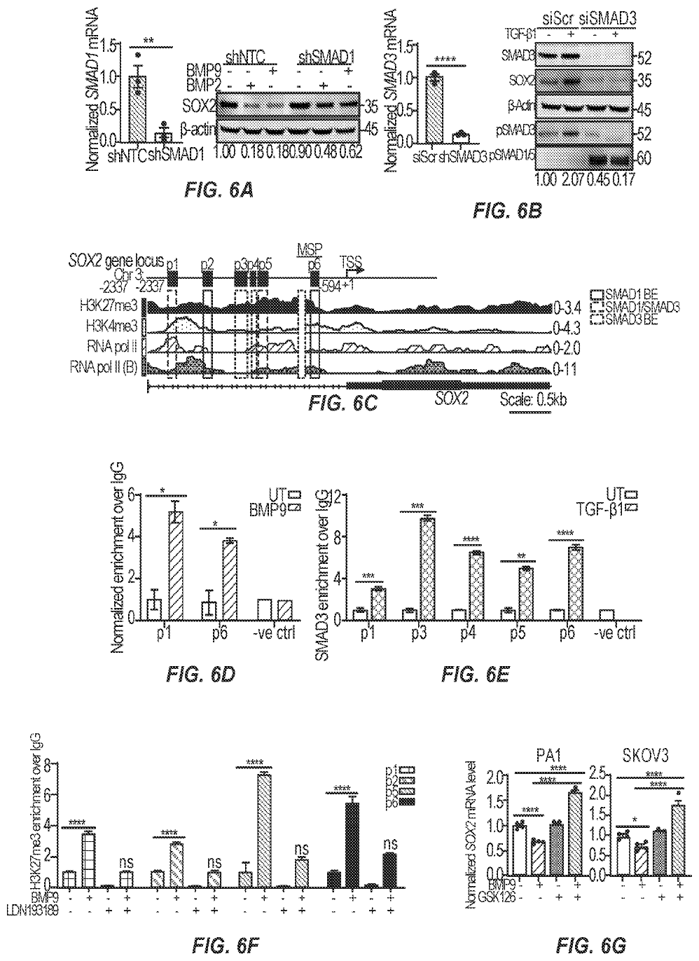

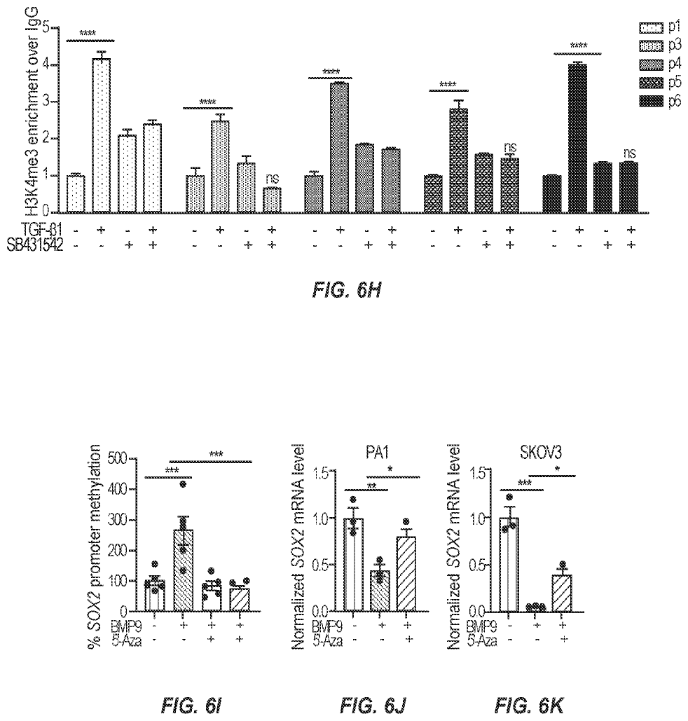

FIG. 6: SMAD1 and SMAD3 directly regulate SOX2 expression and occupy SOX2's promoter at distinct and overlapping sites.

FIG. 6A shows representative qRT-PCR of SMAD1 levels in shSMAD1 cells normalized to shNTC in OVCAR3 cells (left; n=3 technical replicates). Western blot analysis of SOX2 in OVCAR3 shSMAD1 or non-targeting control (shNTC) cells treated with indicated equimolar rhBMPs for 24 hours (right; n=3 biological replicates).

FIG. 6B shows qRT-PCR analysis of SMAD3 in OVCAR3 cells transiently expressing siRNA to SMAD3 (siSMAD3) or scramble control (siScr). Data are normalized to siScr in OVCAR3 cells (left). Western blot analysis of indicated proteins in OVCAR3 siSMAD3 or scramble control (siScr) cells treated with TGF-β1 for 24 hours with quantitation of SOX2 relative to actin shown below (right; n=3 biological replicates).

FIG. 6C shows in silico analysis showing primers flanking SMAD1 and SMAD3-binding elements (“BE”) in chromosomal regions including SOX2's promoter and gene as indicated. TSS, transcription start site; MSP, methylation-specific PCR primer.

FIG. 6D shows relative qRT-PCR of indicated regions (primer sites) after chromatin immunoprecipitation (ChIP) of SMAD1 with or without 1 h of rhBMP9 treatment, expressed as the ratio over IgG controls normalized to untreated cells (n=2 biological replicates).

FIG. 6E shows representative relative qRT-PCR of indicated regions (primer sites) after ChIP of SMAD3 with or without 1 h of TGF-β1 treatment, expressed as the ratio over IgG controls normalized to untreated cells (n=3 technical replicates).

FIG. 6F shows representative qRT-PCR of indicated regions (primer sites) associated with H3K27me3 enrichment with and without 1 hour of rhBMP9 treatment±LDN193189 as indicated in PA1 cells expressed as the ratio over IgG controls normalized to untreated cells (n=3 technical replicates).

FIG. 6G shows qRT-PCR analysis of SOX2 levels in indicated cells pretreated with 5 μM GSK126 for 5 days, followed by treatment with BMP9 for 24 h. Data are normalized to DMSO controls (n=4 biological replicates).

FIG. 6H shows representative qRT-PCR of indicated regions (primer sites) after ChIP with H3K4me3±1-h TGF-β1±SB431542 as indicated in PA1 cells expressed as the ratio over IgG controls normalized to untreated cells (n=3 technical replicates).

FIG. 6I shows MS-qPCR using primers proximal to SOX2's TSS (MSP in C) in PA1 cells pretreated with 5 μM 5′-azacytidine (5′-Aza) for 48 h, followed by treatment with rhBMP9 for 24 h, normalized to DMSO control (n=5 biological replicates).

FIG. 6J and FIG. 6K show qRT-PCR analysis of SOX2 in (J) PA1 and (K) SKOV3 cells treated with 5 μM 5′-Aza and 10 nM rhBMP9, normalized to DMSO control (n=3 biological replicates). Data are presented as the mean±SEM; *p<0.05, **p<0.01, ***p<0.001 (ANOVA followed by Sidak's multiple comparison test).

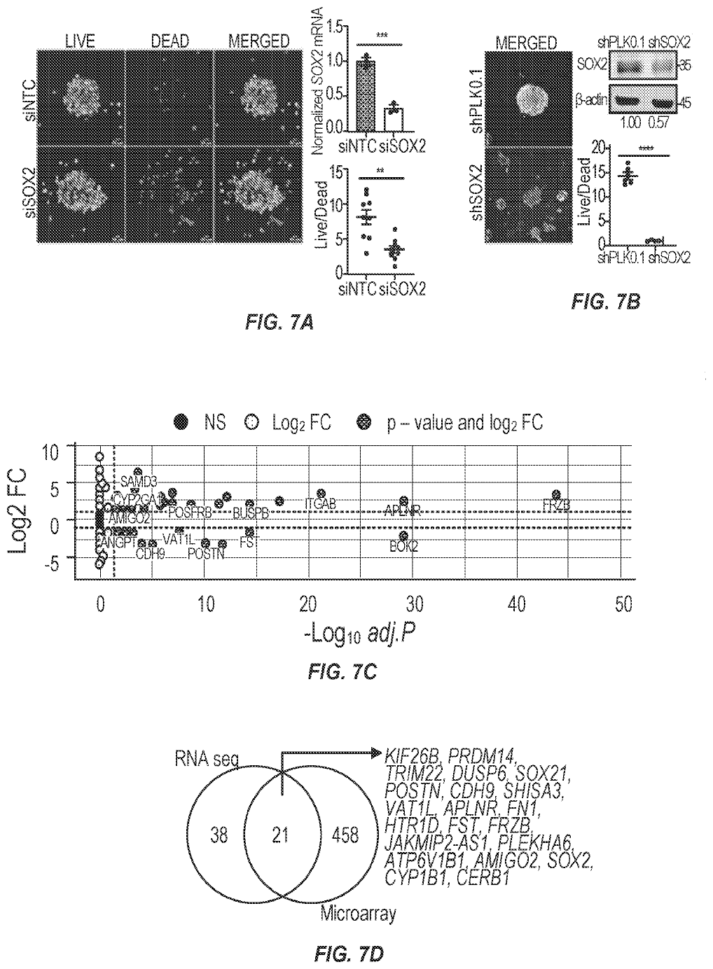

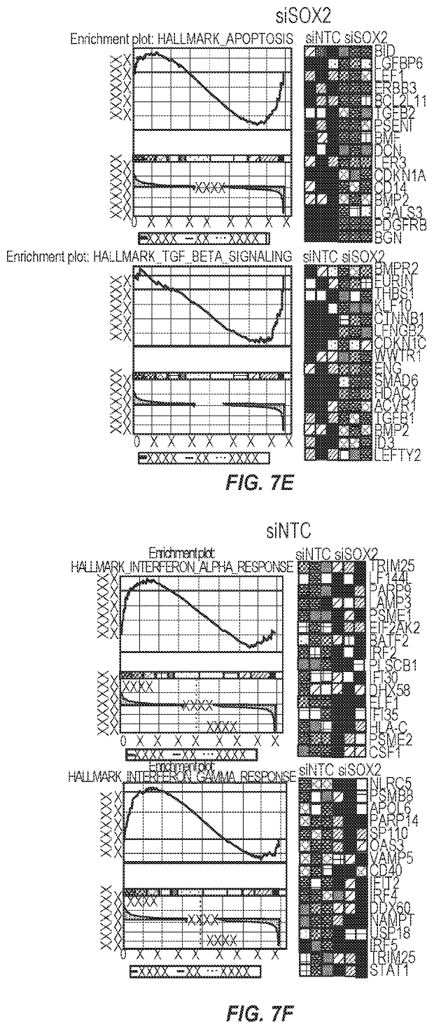

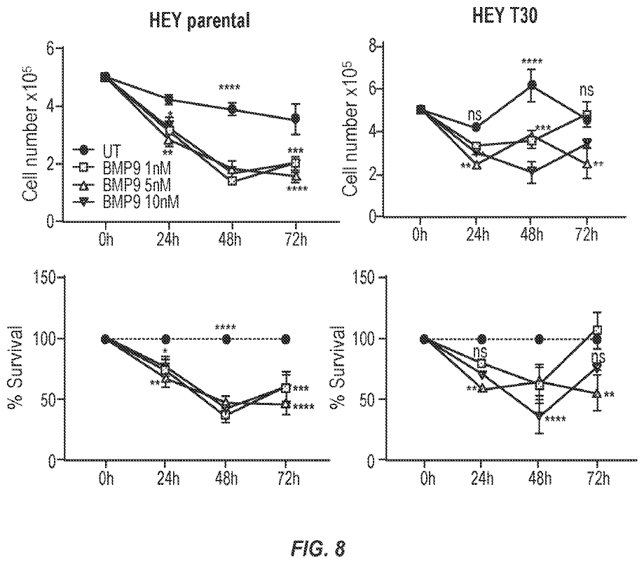

FIG. 7: Genome-wide transcriptome changes upon reducing SOX2 and increasing anoikis reveal apoptotic pathways and key transcriptional epigenetic regulators and adhesion molecules.

FIG. 7A shows representative confocal image from siNTC or siSOX2 PA1 cells under anchorage independence for 72 h (left) with quantitation of live/dead ratio (bottom right, n=9 spheroids/condition. Scale bar, 50 μm). qRT-PCR of SOX2 expression in siSOX2 cells normalized to siNTC cells (top right, n=3 technical replicates).

FIG. 7B shows representative images from PA1 shPLKO.1 and shSOX2 cells under anchorage independence for 72 hours (left). Western blot of SOX2 in shPLK0.1 and shSOX2 cells (top right), and quantitation of live/dead ratio in spheroid cells (n=6 spheroids/condition, bottom right. Scale bar, 50 μm). *p<0.05, **p<0.01, ***p<0.001.

FIG. 7C shows volcano plot of significant DEGs based on adjusted p value of 0.05 between siNTC and siSOX2 in PA1 cells under anchorage independence for 48 hours.

FIG. 7D shows Venn diagram of common DEGs between RNA-seq data from (A) and microarray data from BMP9 treatment under anchorage independence in PA1 cells from FIG. 2A.

FIG. 7E and FIG. 7F show gene set enrichment analysis (GSEA) of pathways differentially altered in (E) siSOX2 and (F) siNTC with corresponding Blue-Pink O'gram of core enrichment genes generated by GSEA (right) (see also FIG. 22).

FIG. 8: Paclitaxel sensitive HEY parental cells are more responsive to rhBMP9 treatment than resistant cells under 3D anchorage independence condition.

FIG. 8(A-B) show kinetics and dose course of indicated rhBMP9 concentration (treatment starting from time 0 hours) on cell survival rate under anchorage independence in (A) HEY parental and (B) HEY T30 cells (n=3 technical trials).

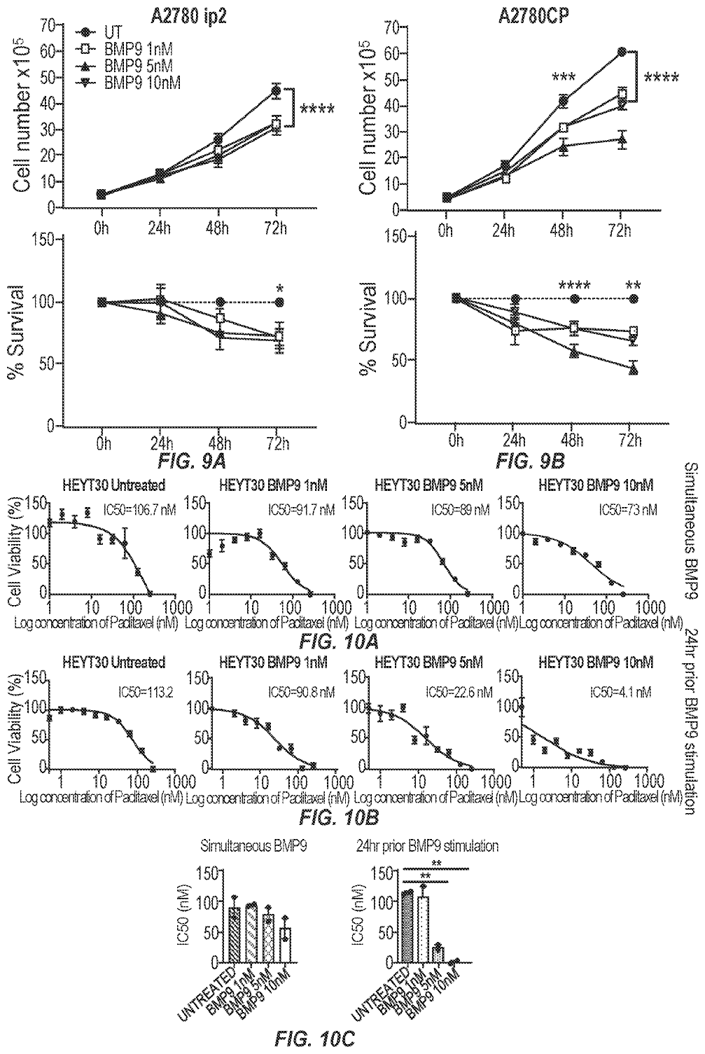

FIG. 9: Cisplatin resistant A2780CP cells are responsive to rhBMP9 treatment under 3D anchorage independence condition.

FIG. 9(A-B) show kinetics and dose course of indicated rhBMP9 concentration (treatment starting from time 0 h) on cell survival rate under anchorage independence in (A) A2780ip2 and (B) A2780CP cells (n=3 technical trials).

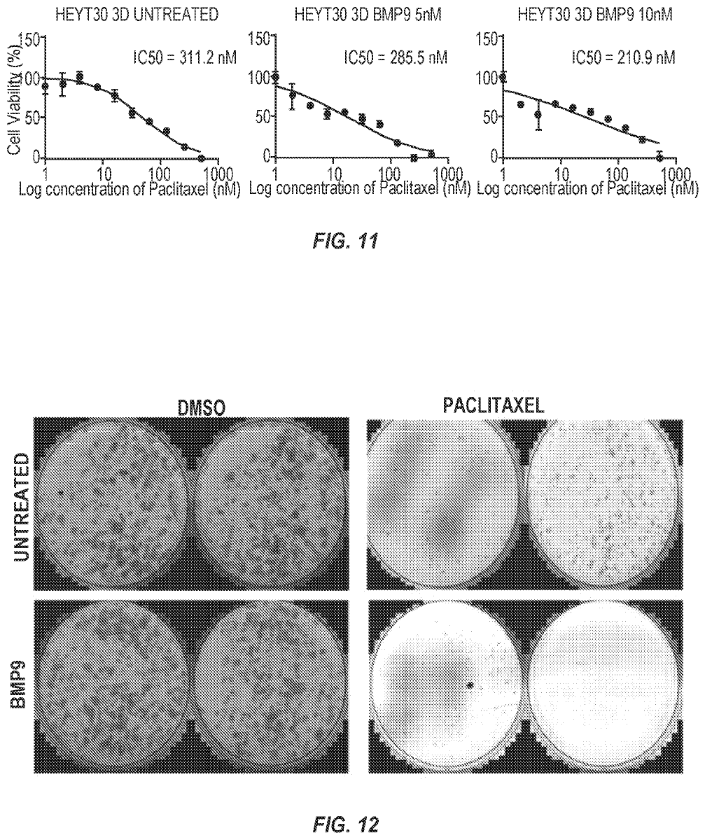

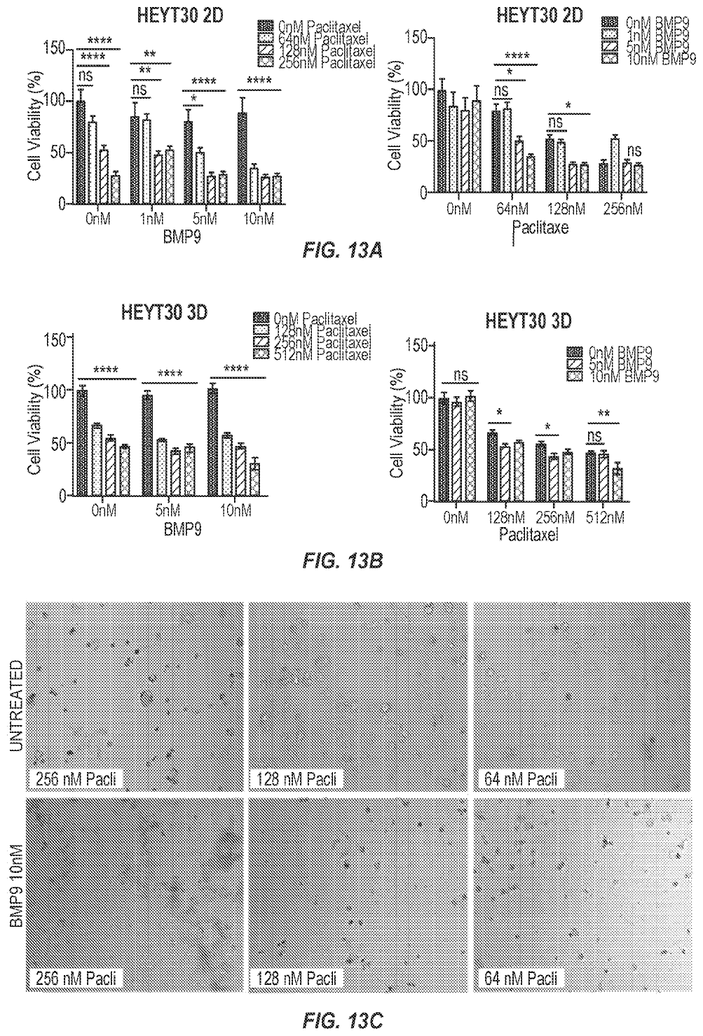

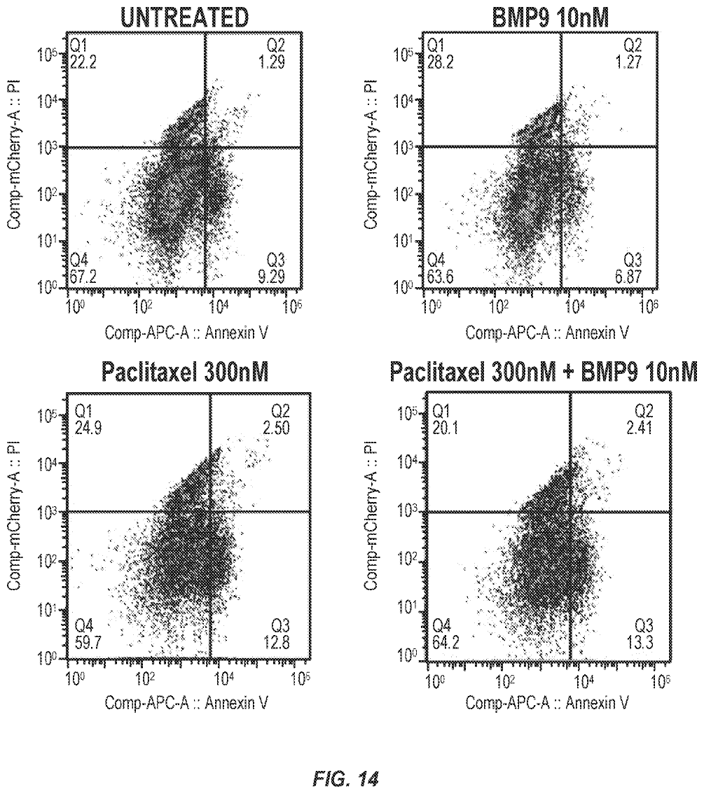

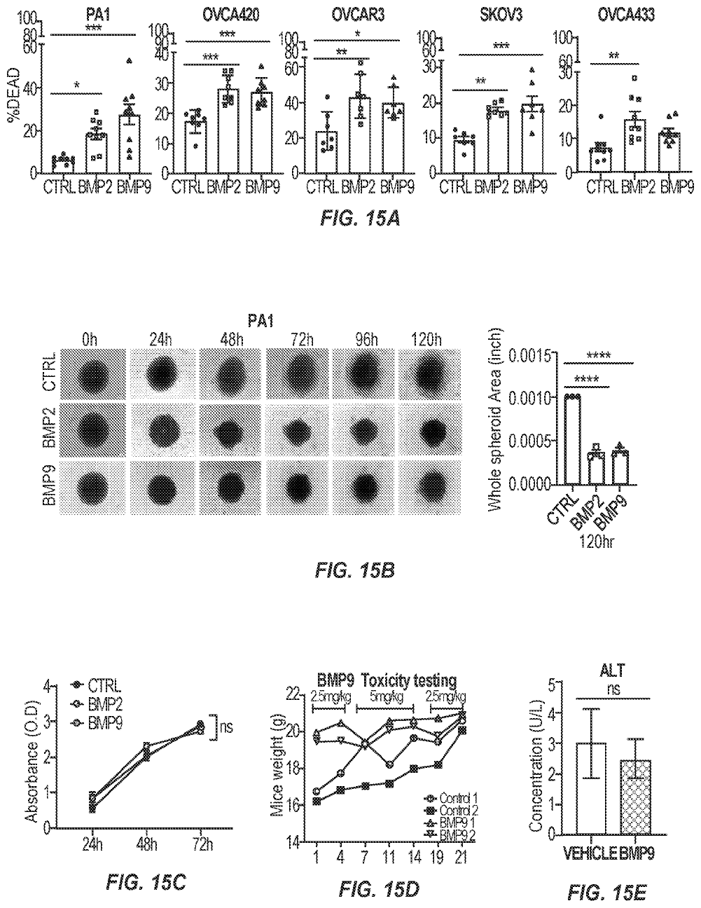

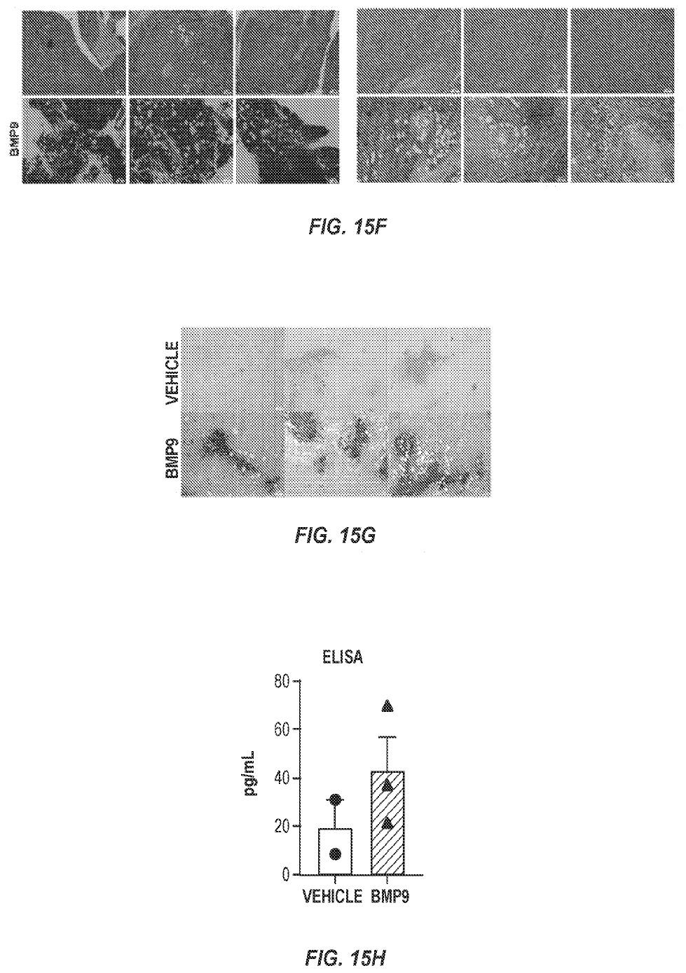

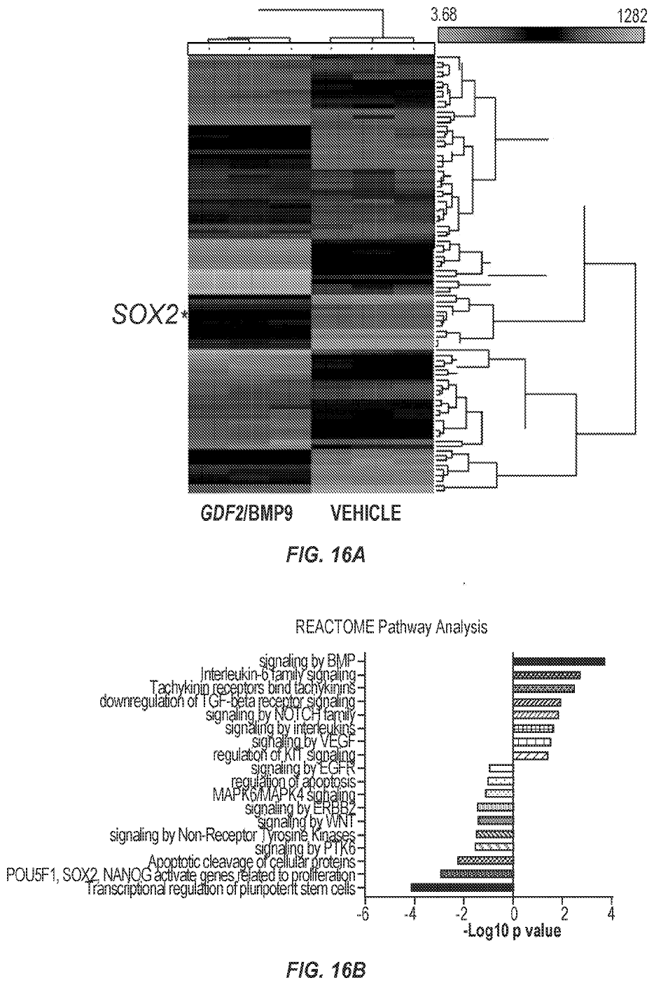

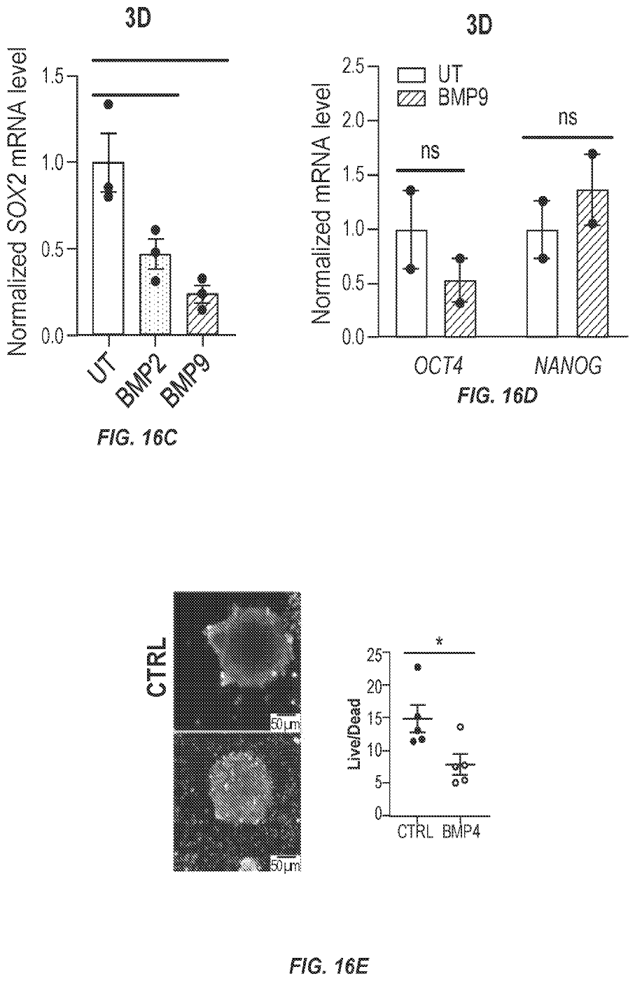

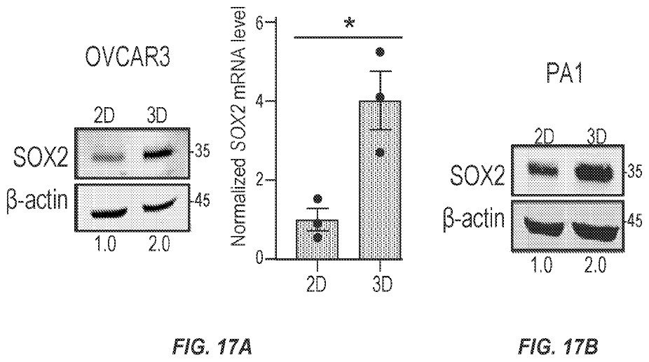

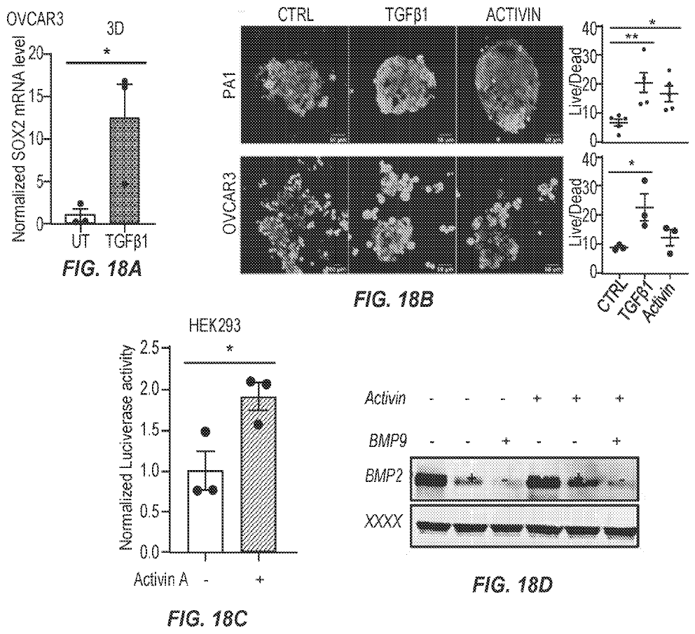

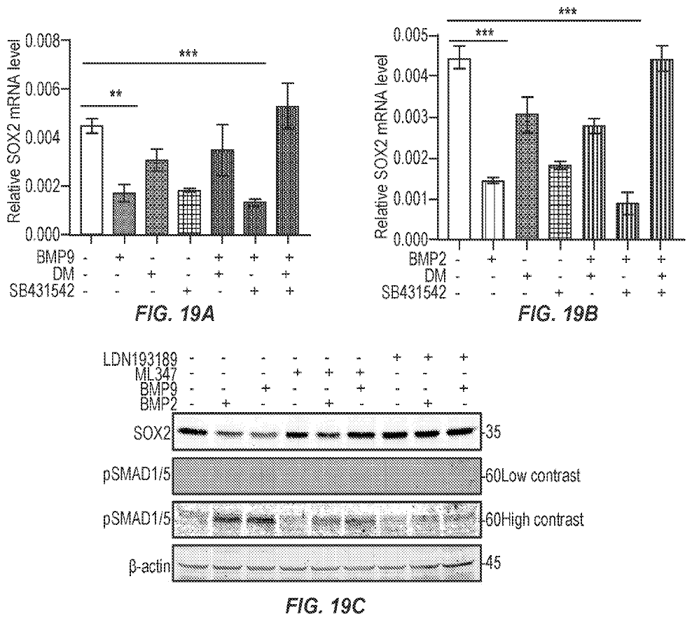

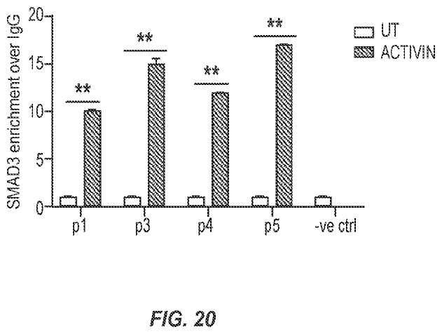

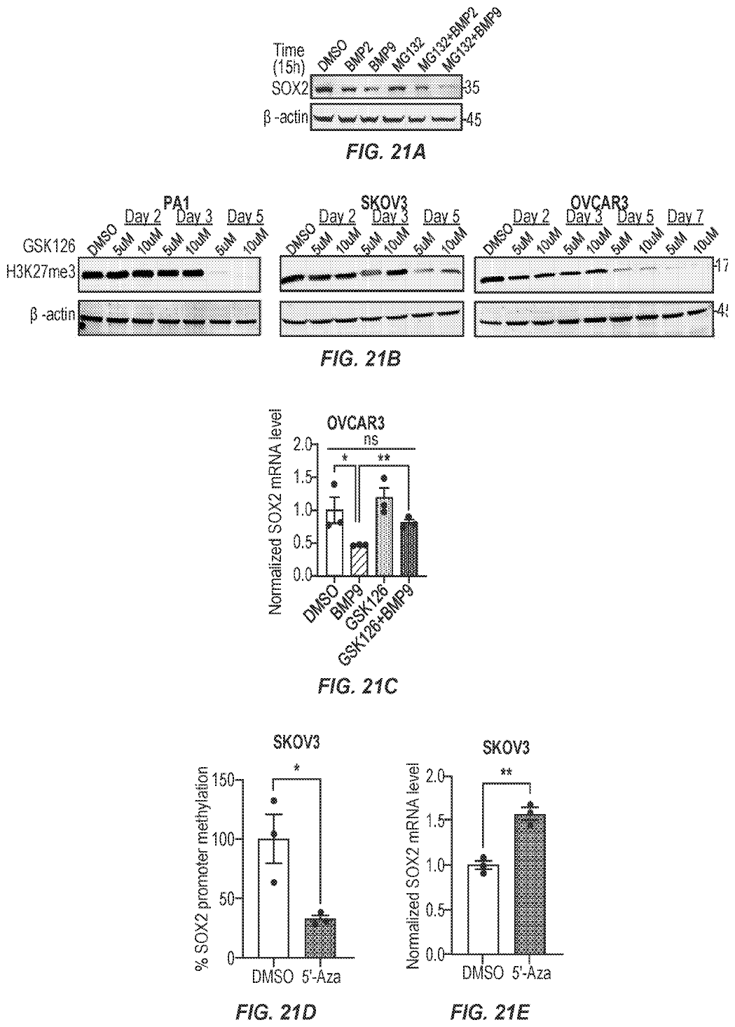

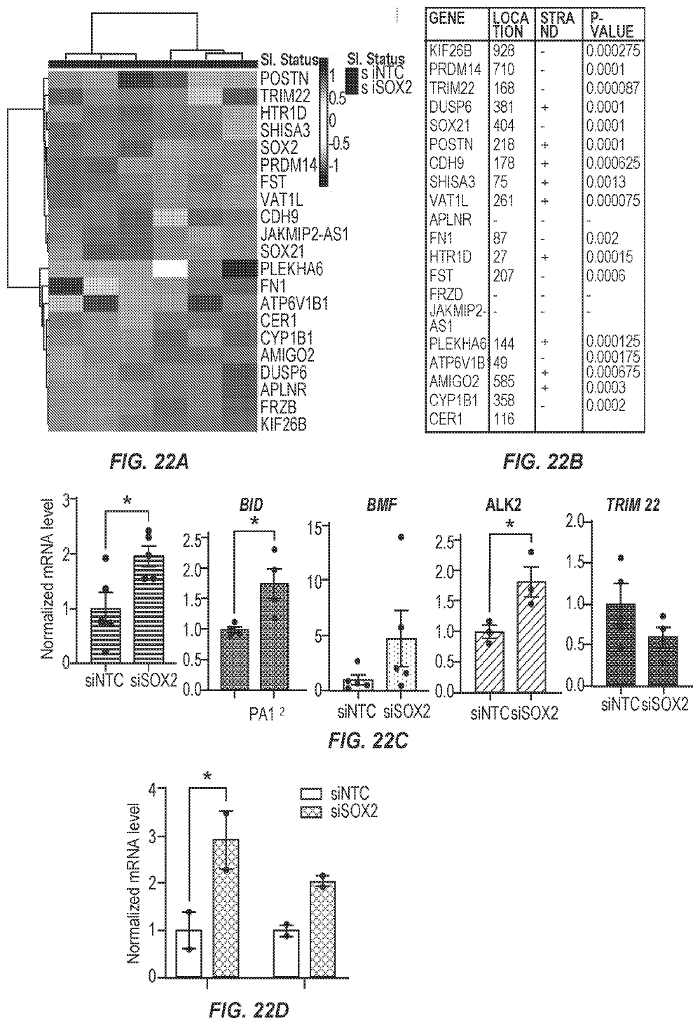

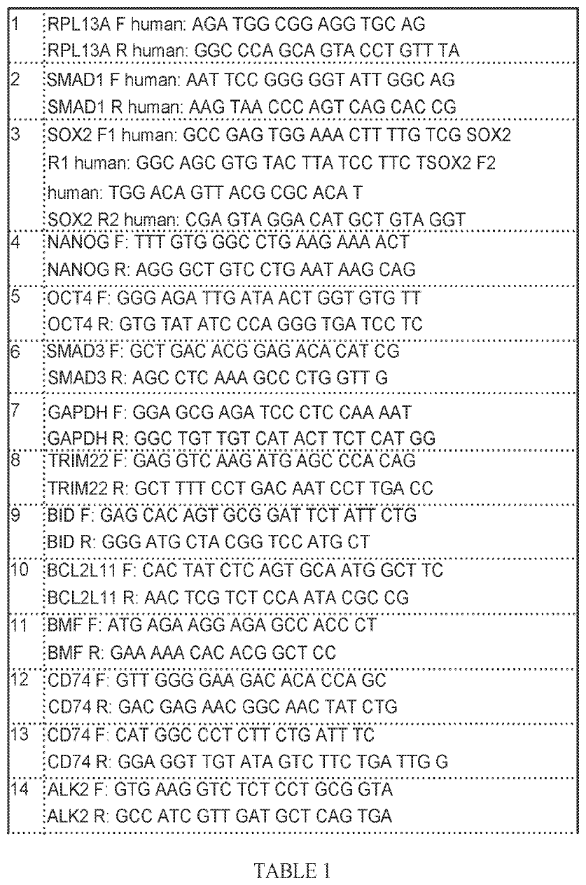

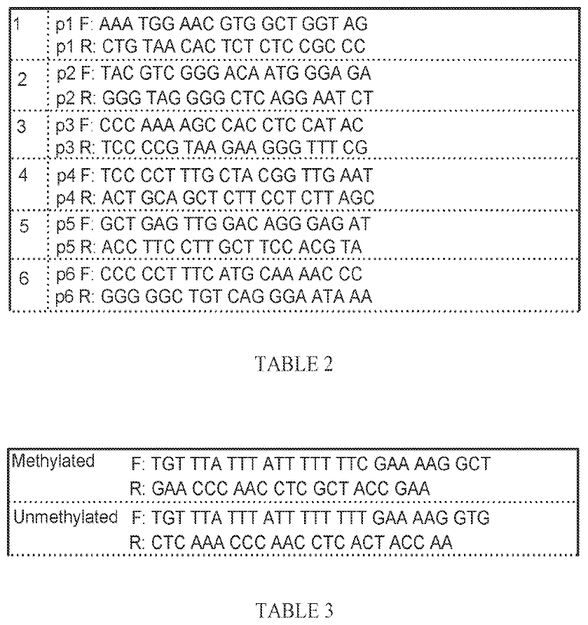

FIG. 10: rhBMP9 increases chemosensitivity of paclitaxel resistant HEYT30 cells. FIG. 10A Cell viability of paclitaxel in HEYT30 simultaneously treated with indicated doses of rhBMP9 for 48 hours.

FIG. 10(B) shows cell viability of paclitaxel in HEYT30 cells with 24 hr prior rhBMP9 stimulation, followed by combination treatment of rhBMP9 and paclitaxel for 48 hours. FIG. 5C Quantification of IC50 of n=2 biological trials for both A and B.

FIG. 11: rhBMP9 increases paclitaxel chemosensitivity under 3D conditions. Cell viability of paclitaxel in HEYT30 treated 24 hours prior with rhBMP9, followed by combination treatment of rhBMP9 and paclitaxel for 48 hours (n=2 biological trials).

FIG. 12: Combinatorial rhBMP9 and paclitaxel treatment decreases colony formation in HEY-T30 cells. Clonogenic assay in HEYT30 cells treated with 80 nM paclitaxel, 10 nM rhBMP9 or combination for 7 days. Colonies were stained with crystal violet solution and imaged (n=2 biological trials).

FIG. 13: rhBMP9 potentiates paclitaxel to induce cytotoxicity in paclitaxel-resistant HEYT30 cells.

FIG. 13(A-B) show co-treatment with indicated dose of rhBMP and paclitaxel in HEY-T30 cells with 24 hours prior 10 nM rhBMP9 stimulation, followed by combination treatment of rhBMP9 and paclitaxel for 48 hours under 2D (A) and 3D (B) conditions (n=2 biological trials).

FIG. 13C shows a pictorial representation of HEY-T30 Cell treated with indicated dose of paclitaxel with 24 hours prior 10 nM rhBMP9 stimulation, followed by combination treatment of BMP9 and paclitaxel for 48 hours (n=2 biological trials).

FIG. 14: Combinatorial treatment increases cell apoptosis in HEYT30 cells. Flow cytometry analysis in HEY-T30 cells treated with 10 nM rhBMP9, 300 nM paclitaxel or combination under 3D conditions for 48 hours, followed by staining with PI and annexin-V (n=2 biological trials).

FIG. 15: The effect of rhBMP9 on cell viability in vitro and in vivo is shown.

FIG. 15A shows percent dead cells in OVCA cell lines from FIG. 1A cultured under anchorage independence for 48 hours, and subsequently treated with either vehicle (VEH) control or with 10 nM rhBMP2 or rhBMP9 for 24 hours (n=7-10 spheroids/condition).

FIG. 15B shows 3D matrigel invasion assay of spheroids in the presence of

control, rhBMP2 or rhBMP9 (10 nM) (left). quantitation of spheroid invasion for the indicated time in PA1 cells (right) (n=3 spheroids/condition).

FIG. 15C shows growth curve of PA1 cells grown under attached 2D conditions in the presence of control, rhBMP2 or rhBMP9 (n=3 biological replicates).

FIG. 15D shows body weight in grams of NOD-SCID mice receiving either vehicle or rhBMP9 at indicated doses for a 21 days period (n=2 mice per group).

FIG. 15E shows Alanine Transaminase (ALT) concentration as a measure of liver function measured from plasma (n=2 mice per group).

FIG. 15F shows additional representative images showing complete omental infiltration in vehicle receiving mice as compared to rhBMP9 receiving mice injected with PA1-luc-GFP (left) and SKOV3-luc-GFP (right) (n=2 mice per group).

FIG. 15G shows representative necrotic region (dark brown) in tumors from rhBMP9 vs vehicle receiving mice injected with SKOV3-luc-GFP (Scale bar=50 μM) (n=2 mice per group). H ELISA of rhBMP9 in plasma from mice in vehicle and rhBMP9 treated groups in PA1-luc-GFP mice (n=2-3 mice per group). Data are presented as mean±SEM, *p<0.05, **p<0.01, ***p<0.001. Statistical significance is determined using (A-B) ANOVA followed by Dunnett's multiple comparisons test. (C-H) unpaired Student's t-test.

FIG. 16: Effect of indicated rhBMPs on gene expression, SOX2 and anoikis.

FIG. 16A shows a heatmap of transcription profile of 48,226 genes in PA1 cells treated with BMP9 for 24 hrs under anchorage independence (n=3 biological replicates).

FIG. 16B shows REACTOME pathway analysis of genes from data in (A).

FIG. 16C shows qRTPCR analysis of SOX2 after rhBMP treatment for 24 hrs under anchorage independence (3D) in PA1 cells (n=3 biological replicates).

FIG. 16D shows qRT-PCR analysis of OCT4 and NANOG after rhBMP9treatment under 3D condition in PA1 cells (n=2 biological replicates).

FIG. 16E shows representative images of PA1 cells cultured under anchorage independence for 48 hours, and subsequently treated with either vehicle control or with 10 nM rhBMP4 for 24 hrs. Live/dead cell ratios were assessed by staining with Calcein-AM and Ethidium homodimer dye. Scale bar =50 μm (n=5 spheroids/condition).

FIG. 16F shows representative images of PA1 cells cultured under anchorage independence for 48 hours, and subsequently treated with either vehicle control or with 10 nM rhBMP10 for 24 hrs (n=7-8 spheroids/condition).

FIG. 16G shows qRT-PCR analysis of SOX2 in tumors from vehicle and rhBMP9 treated groups in SKOV3-luc-GFP mice (n=5 replicates from 2 mice per condition). Data are presented as mean±SEM, *p<0.05, **p<0.01, ***p<0.001; Statistical significance is determined using (C-D) ANOVA followed by Dunnett's multiple comparison test; and (E-G) unpaired Student's t test.

FIG. 17: Alterations in SOX2 levels in attached growth (2D) versus in suspension (3D).

A Western blot (left) and normalized qRT-PCR (right) of SOX2 expression under attached (two-dimensional, “2D”) versus under anchorage independence (three-dimensional, “3D”) conditions in OVCAR3 cells after 72 hrs under 3D condition. (Western blot n=4 biological replicates; qRT-PCR n=3 biological replicates). B Western blot of SOX2 expression under 2D versus 3D condition in PA1 cells after 72 hrs under 3D condition (Western blot n=2 biological replicates). Data are presented as mean±SEM, *p<0.05, **p<0.01, ***p<0.001. Statistical significance is determined using unpaired t-test.

FIG. 18: Effect of TGF-β1 and activin A on anoikis and SOX2 promoter luciferase activity, and the effect of ligand combination on SOX2 levels.

FIG. 18A shows the relative qRT-PCR analysis of SOX2 after TGF-β1 treatment for 24 hours under anchorage independence (3D) condition in OVCAR3 cells (n=3 biological replicates).

FIG. 18B shows live-dead analysis of cells under anchorage independence after (1 nM) TGFβ1 and (10 nM) activin treatment for 24 hrs in indicated cells (n=3-5 spheroids/condition).

FIG. 18C shows pGL3-SOX2 promoter-reporter luciferase analysis upon 10 nM activin A treatment for 24 hours in indicated cells normalized to untreated and renilla internal control (n=3 biological replicates).

FIG. 18D shows Western blot of SOX2 after combined treatment of equimolar (10 nM) activin and rhBMP2/9 for 24 hrs in PA1 cells (n=2 biological replicates). Data are presented as mean±SEM, *p<0.05, **p<0.01, ***p<0.001. Statistical significance is determined using (A-C) unpaired t test, B ANOVA followed by Sidak's multiple comparisons test.

FIG. 19: Effects of ALK receptors inhibition on SOX2 levels in OVCAR3 cells.

FIG. 19A shows qRT-PCR analysis of SOX2 expression in OVCAR3 cells pretreated with 5 μM Dorsomorphin (DM) and 5 μM SB431542 for 1 hr, followed by treatment with rhBMP9 for 24 hrs. (Data are normalized to DMSO vehicle controls and presented as mean±SEM for three technical replicates).

FIG. 19B shows qRT-PCR analysis of SOX2 in OVCAR3 cells pretreated with 5 μM Dorsomorphin (DM) and 5 μM SB431542 for 1 hr, followed by treatment with rhBMP2 for 24 hours. (Data are normalized to DMSO vehicle controls and presented as mean±SEM for three technical replicates).

FIG. 19C shows Western blot of SOX2 levels in OVCAR3 cells pretreated with 3 μM ALK1,2 inhibitor ML347 and 0.8 μM ALK2,3 LDN193189 for 1 hr, followed by treatment with BMP2/9 for 24 hours. (n=2 biological replicates).

19A-B shows data presented as mean±SEM. *p<0.05, **p<0.01, ***p<0.001. Statistical significance is determined using t-test.

FIG. 20: ChIP of SMAD3 in response to activin A. Representative relative qRT-PCR of indicated regions (primer sites) after chromatin immunoprecipitation of SMAD3 to sites on SOX2 proximal chromosomal regions with or without 1 hr of activin A treatment, as indicated in PA1 cells expressed as the ratio over IgG controls normalized to untreated cells. Data is presented as mean±SEM for 3 technical replicates. *p<0.05, **p<0.01. Statistical significance is determined using Student's t-test.

FIG. 21: Effect of inhibiting the proteosome, histone methylation and DNA methylation on BMP mediated SOX2 repression.

FIG. 21A shows Western blot of SOX2 levels in PA1 cells pretreated for 1 hour with 0.5 μM MG132, followed by rhBMP2/9 (n=2 biological replicates).

FIG. 21B shows time and dose course of the effect of GSK126 on H3K27me3 levels in indicated cell lines (n=2 biological replicates).

FIG. 21C shows qRT-PCR analysis of SOX2 expression in cells treated with 10 nM BMP9 and 5 μM GSK126 for 7 days in OVCAR3 (n=3 biological replicates).

FIG. 21D shows MS-qPCR analysis of SOX2 in SKOV3 cells pretreated with or without 5 μM 5′-Aza for 48 hours, normalized to DMSO control (n=3 biological replicates).

FIG. 21E shows qRT-PCR analysis of SOX2 in SKOV3 cells treated with or without 5 μM 5′-Aza, normalized to DMSO control (n=3 biological replicates). Data are normalized to DMSO vehicle controls. Data are presented as mean±SEM, *p<0.05, **p<0.01, ***p<0.001. Statistical significance is determined using the t-test.

FIG. 22: Transcriptomic analysis of SOX2 silencing during anchorage independent survival (suspension cultures).

A Heatmap of 21 common differentially expressed genes between the microarray from GEO Database: #GSE185924, FIG. 2A and RNA sequencing analysis GEO Database: GSE185932, FIG. 7C. B LASAGNA analysis of SOX2 binding motifs for the 21 DEGs from (A). C-D qRT-PCR analysis of indicated candidate genes in siNTC and siSOX2 normalized to siNTC in either PA1 cells or OVCAR3 as indicated. (n=3-5 biological replicates). Data are presented as mean±SEM, *p<0.05, **p<0.01, ***p<0.001. Statistical significance is determined using (C) unpaired the t-test and (D) ANOVA followed by Sidak multiple comparison test.

Publications cited herein and the material for which they are cited are hereby specifically incorporated by reference in their entireties.

In the following, further embodiments are described to facilitate the understanding of the invention:
Embodiment 1: A method for reducing metastasis in a subject with cancer, the method comprising administering an effective amount of BMP9 or an agonist thereof to the subject with cancer.
Embodiment 2: The method according to Embodiment 1, wherein the BMP9 is recombinant BMP9 (rBMP9).
Embodiment 3: The method according to Embodiment 1 or 2, wherein the cancer is selected from the group consisting of breast cancer, lung cancer, gynecological cancer, neuroendocrine cancer, and combinations thereof.
Embodiment 4: The method according to Embodiment 1 or 2, wherein the cancer is gynecological cancer, such as ovarian cancer.
Embodiment 5: The method according to any one of Embodiments 1-4, wherein the BMP9 is rhBMP that is administered daily for a period of at least 7 days.
Embodiment 6: The method according to any one of Embodiments 1-5, wherein the BMP9 is rhBMP that is administered in an amount from 0.01 mg/kg to 50 mg/kg,
Embodiment 7: The method according to any one of Embodiments 1-6, wherein the subject is a mammal.
Embodiment 8: The method according to any one of Embodiments 1-7, wherein the subject is a human.
Embodiment 9: The method according to any one of Embodiments 1-8, wherein administering the BMP9 or an agonist thereof suppresses three dimensional spheroid cell invasion.
Embodiment 10: The method according to any one of Embodiments 1-9, wherein the BMP9 or an agonist thereof is administered in a pharmaceutical composition comprising the BMP9 or an agonist thereof and a pharmaceutically acceptable carrier.
Embodiment 11: The method according to Embodiment 10, wherein the pharmaceutical composition further comprises an additional therapeutic agent.
Embodiment 12: The method according to Embodiment 11, wherein the additional therapeutic agent is an anti-cancer compound.

### EXAMPLES

### Methods

### Mice

All animal studies were performed in accordance with the Institutional Animal Care and Use Committee at the University of Alabama Birmingham. Female SCID mice (Key resources table) at 5-7 weeks of age were housed under pathogen-free conditions at the Animal Research Facility at UAB. 1.5×106 GFP-luciferase SK-OV3 cells or 3×106 GFP-luciferase expressing PA1 cells were intraperitoneally injected. Mice were monitored daily, with girth and weight measurement taken twice a week. Tumor progression was tracked weekly using the IVIS Lumina III In vivo Imaging System (Caliper Life Sciences, MA) at UAB's Small Animal Imaging Facility. rhBMP9 was administered at the time of tumor cell injection followed by daily intraperitoneal (i.p.) injections of 1 mg/mL in 4 mM HCl+PBS+DI water (Vehicle), to achieve a final 5 mg/kg dose. For metastasis and tumor growth analysis, mice were euthanized between 21-50 days depending on the cell line. At necropsy, ascites, if present, were collected and volumes measured when possible, tumor weights in the omentum and other organs were recorded and collected when possible. For survival studies, mice that reached end-point criteria, including continued weight loss, respiratory trouble and permanent recumbency were euthanized. For microscopic analysis of tissues, formalin-fixed tissues were processed, paraffin-embedded, and sectioned at 5 μm thickness and H&E stained at UAB's histology core.

### Cell Lines, Antibodies and Reagents

Authentication was carried out at UAB's Heflin Center for genomics by STR profiling. Human cell lines were culture in RPMI-1640 (ATCC® 30-2001™) containing L-glutamine, 10% fetal bovine serum (FBS), and 100 U of penicillin-streptomycin except OVCAR3, which were cultured with 20% FBS. Patient ascites derived EOC15 and AF68cells were culture in 1:1 MCDB 105 and MCDB 131 with 15% FBS. HEK293 cells were maintained in complete DMEM supplemented with L-glutamine, 10% FBS, and penicillin-streptomycin. All cell lines were maintained at 37° C. in a humidified incubator at 5% CO2, routinely checked for mycoplasma (MycoAlert PLUS mycoplasma detection kit, Lonza, Basel, Switzerland), and experiments conducted within 3-6 passages of testing depending on the cell line. Luc-GFP cell lines were generated using pHIV-Luc-ZsGreen construct. PA1 and SKOV3 cells were transduced followed by cell sorting at the UAB Flow Cytometry Core to generate stable PA1-Luc-GFP and SKOV3-Luc-GFP cells.

### RNA Interference and Over-Expression

Sequences for all constructs and primers are in Tables 1, 2 and 3. Lentiviral particles were generated as previously described (Varadaraj et al., 2015). For stable SMAD1 knockdown, cells were infected with a pool of three individual SMAD1 shRNA lentivirus or non-targeting control (NTC) constructs in complete RPMI media. The media was changed after 24 hours to fresh RPMI supplemented with 10% FBS and 13PS and left for an additional 48 hours. siRNA-mediated knock-down of SMAD3 and SOX2 was achieved using a pool of two independent siRNA duplexes to SMAD3 or SOX2, respectively and a scrambled siRNA duplex used as a negative control. Transfection was performed using Lipofectamine RNAimax reagent. Briefly, 1 3 105 cells were cultured in 6 well plates in full serum medium for 24 hours. Medium was replaced with 1 mL Opti-MEM, containing 10 nM siRNA duplexes and 7.5 mL Lipofectamine RNAi-max. After 15-24 hours, 1 mL 10% serum medium was added to the cells and incubated for 72 hours. The knockdown was confirmed by qRT-PCR (see in Table 1) and/or western blotting. For adenovirus infection, cells were infected with 100 MOI of adenovirus construct expressing ALK2 (Q-D)-HA, ALK3 (Q-D)-HA, generously provided by Gerard C. Blobe and Miyazono K. Transient DNA transfections were carried out in PA1 cells using Lipofectamine LTX dissolved in Opti-MEM medium. For SOX2 overexpression cell lines, indicated cells were infected with EFIA-SOX2 and LV-CMV-SOX2 lentivirus and their respective controls (see Table 1) independently in complete RPMI media with polybrene for 24 hours per instructions. The media was then changed to fresh growth media and incubated for 48 hours, followed by puromycin selection.

### Anchorage-Independence Suspension Anoikis Assays

For Live/Dead analysis under anchorage independence, 1,000 cells were seeded in 96-well hydrogel-coated ultra-low attachment (ULA) plates (Corning #4515) for times indicated. Growth factor treatment was done under low serum 2.5% FBS condition (Cells were plated in 10% FBS to allow spheroid formation before beginning treatment). Cells were then stained with 2 μM Calcein-AM and 4 μM Ethidium-homodimer for 30 min before imaging. z-stacked images were obtained using the Zeiss LSM700 Confocal microscope (Microscopy and Flow Cytometry Core, University of South Carolina) and NIKON A1 Confocal microscope (UAB High Resolution Imaging Facility). Fluorescent quantification was performed using ImageJ Fiji software to calculate the Corrected Total Cell Fluorescent (CTCF)=Integrated Density−(Area of selected cell×Mean fluorescence of background readings) per spheroid.

Lysate and RNA preparation: 100,000-300,000 cells were seeded in a poly-HEMA coated 6-well plate for indicated times in full serum unless indicated otherwise. Cells were collected by centrifugation and lysed with trizol for RNA extraction or direct 2× lysis buffer for protein lysates.

### 3D Spheroid Invasion

Spheroid invasion through Matrigel was performed as previously described (Vinci et al., 2015) using 1,000 cells that were allowed to form spheroids in ULA wells for 72 hours. Specifically, Matrigel was mixed with BMP2 and BMP9 to ensure a final concentration of 10 nM and added gently to the spheroids in the well, then incubated at 37° C. for 1 hour to allow Matrigel solution to solidify. Additional growth media with BMP was added to each well as the top layer. Invasion was then monitored for up to 120 hours. Quantification of the amount of spread/invasion was done using ImageJ software. Sulforhodamine B (SRB) growth assay

Growth of cells was monitored by seeding 1,000 cells per well in a 96 well plate, followed by treatment with 10 nM BMP2/9 for indicated times. At endpoint, medium was shaken out from plates, followed by addition of cold 10% trichloroacetic acid in each well, and incubation at 4° C. for 10 minutes. Five sequential wash steps were performed by complete immersion in water and shake out of the water from the plate. Next, 0.4% sulforhodamine B (SRB) in 1% acetic acid was added to each well and incubated at room temperature for 10 minutes. Five washed were done by complete immersion in 1% acetic acid followed by complete removal of the remaining wash. Plates were air dried overnight. Finally, 200 mL of 20 mM Tris pH 10 (unbuffered) was added to each well and plate rocked on a rotary shaker for 1-2 hours and absorbance measured at 570 nm with a plate reader (Vichai and Kirtikara, 2006).

### Immunohistochemistry (IHC) and TUNEL Assay

IHC was performed using the BioCare Mach4 Universal Detection Kit. Specifically, anti-SOX2 was diluted in Da Vinci Green Diluent and incubated overnight at 4° C. in a humidified chamber. HRP was detected with 3,30-diaminobenzidine (DAB) substrate for 4 minutes. TUNEL staining was performed according to the manufacturer's instruction. Slides were examined and images captured with EVOS M7000 microscope. Cell profiler (Stirling et al., 2021) and Image J Fiji software were used for image quantification.

Microarray and RNA sequencing

Total cellular RNA was extracted using Trizol reagent according to the manufacturer's protocol. RNA quality was determined using an Agilent 2100 Bioanalyzer and an RNA 6000 Nano kit (Agilent, Cat. No. 5067-1511) with RNA integrity numbers (RIN) ranging from 9.8 to 10. Microarray analyses were performed on the GeneChip™ Human Gene 2.0 ST ArrayS (Thermo Fisher Scientific, Cat. No. 902112) by the functional genomic core at University of South Carolina. Data were imported into the Affymetrix GeneChip Expression Console 1.4.1.46 and processed at the gene-level using the Robust Multichip Analysis (RMA) algorithm to generate CHP files. Experimental-group specific transcriptional responses were determined using unpaired one-way between-subject analysis of variance (ANOVA). Differentially expressed genes with p-values smaller than 0.05 and fold change higher than 2.0 and lower than −2.0 were used for further bioinformatics analysis.

For RNA sequencing: library preparation was performed on purified, extracted RNA using a KAPA mRNA HyperPrep Kit (Kapa, Biosystems, Wilmington, MA) according to the manufacturer's protocol. High throughput sequencing with 75-bp single-end reads was performed on an Illumina NextSeq 550 using an Illumina NextSeq 500/550 High Output Kit. Reads were aligned to the human transcriptome GENCODE v35 (GRCh38.p13) using STAR and counted using Salmon (Dobin et al., 2013; Patro et al., 2017). Normal-ization and differential expression analysis were performed using the R package DESeq2 (Love et al., 2014). Genes where there were fewer than three samples with normalized counts less than or equal to five were filtered out of the final data set. Benjamini-Hochberg-adjusted p-value of p<0.05 and log 2 fold change of 1 were the thresholds used to identify differentially expressed genes between treatment conditions.

### Primary EOC and Patient Ascitic Fluid ELISAs

For cells from patient ascites with an initial diagnosis of high-grade serous adenocarcinoma were collected after informed consent at the Pennsylvania State University College of Medicine (Hershey, PA) or the University of Alabama Birmingham, with approval for the study granted from the Penn State College of Medicine and UAB Institutional Review Boards (IRB). Epithelial cancer cells were isolated from ascites, as previously described (Kim et al., 2020) and used to derive EOC15 and AF68 cells respectively. AF68 was subsequently determined to favor an upper GI primary tumor with a less likely gynecological origin. For the ELISA study, ascites from patients with a confirmed diagnosis of primary OC were analyzed. Ascitic fluid was collected and banked after informed consent at Duke University Medical Center, with approval for the study from Duke University's IRB. Single plex ELISAs were carried out for TGF-β1 and TGF-β2 using Aushon Biosystems Custom Circa Chemiluminescent Array kit while BMP9 was detected as described previously (Liu et al., 2018).

### Luciferase Assay

HEK293 cells were transfected with the pGL3-SOX2 promoter-luciferase reporter plasmid construct and SV40-renilla for 24h. Treatment with BMP2 or BMP9 or TGF-or Activin A was carried out for 24 hours in serum-free media at either 10 nM or 400 pM respectively. According to the manufacturer's instruction, cells were collected and lysed in 1 3 passive lysis buffer. To measure luciferase activity, 20 mL of lysate was added to 25 mL of dual Luciferase Assay Reagent, and luminescence was quantitated using a luminometer (Biotek).

### Chromatin Immunoprecipitation (ChIP) Assay

ChIP was carried out using a modified version of a previously described protocol (Medeiros et al., 2009). Cells were grown to 80% confluency in 150 mm culture dishes. Cells were fixed at room temperature in 1% Paraformaldehyde solution (dilute 8% PFA in serum free media to get 1%) and rocked for 10 minutes. 10×Glycine was added to the plate and allowed to sit for 5 minutes at room temperature. Cells were scraped down and cell suspension were transferred to a cold centrifuge tube for centrifugation at 720 RCF at 4° C. for 10 minutes. Cells were rinsed with 1X Phosphate buffer saline and centrifugation repeated. Cell pellet was next resuspended in lysis buffers described in (Medeiros et al., 2009) to obtain nuclei pellet. This was followed by chromatin sonication, using QSonica sonicator (model CL-188) for four cycles (30% amplitude for 15 secs ON and 30 secs OFF) to obtain DNA fragments with a length from 150 to 300 bp. 1/10th of the supernatant was stored as input control. ChIP was performed using Protein A magnetic beads (Dynabeads, Invitrogen #10001D) to couple 3.5 mg ChIP-grade antibodies for SMAD1, SMAD3, H3K27me3, H3K4me3, or rabbit IgG antibody overnight at 4□C. DNA was purified using the PureLink Quick PCR Purification kit (Thermo Fisher Cat #: K310001) and enrichment of DNA fragments analyzed via relative quantitative RT-PCR (qPCR) using ChIP primers (see Table 2) to specific locations. Negative and positive control regions were included in all analysis.

### Methylation-Specific Quantitative RT PCR

Genomic DNA was extracted, and bisulfite conversion was performed on 500 ng of gDNA using the MethylAmp DNA modification kit according to manufacturer's instructions. Relative quantitative RT-PCR (qPCR) was performed with methylation-specific and unmethylation-specific primers (see Table 3).

### CellTiter Glo 3D Cell Viability Assay

3,000 cells were plated in white Ultra-low attachment (ULA) cell culture plates and treated with BMP9 for 24 hours prior to combination treatment with paclitaxel or simultaneous BMP9 and paclitaxel treatment for 48 hours. The paclitaxel dose range is 0-512 nM or DMSO control, BMP9 dose is an indicated in the legends. This was then followed by performing the CellTiter Glo 3D viability assay according to product manual instruction.

### Clonogenic Survival Assay

500 cells were seeded in 6-well plates, with 24-hour pretreatment with 10 nM BMP9. This was followed by a treatment with the indicated dose of paclitaxel and BMP9 alone or in combination for 8 days. Cells were gently washed with 1×PBS followed by 0.05% crystal violet solution for 2 hours at room temperature. Colonies were captured with the EVOS M7000 microscope and Image J Fiji software was used for image quantification.

### Flow Cytometry

700,000 cells were seeded in 60 mm poly-hema coated culture dishes, followed by incubation with 300 nM paclitaxel and 10 nM BMP9 alone or in simultaneous combination for 48 hours. Cells were centrifuged and resuspended in 1×binding buffer, followed by incubation with Annexin V and PI according to the manufacturer's procedure manual. The stained cell suspension was added to FACS tubes containing 200 μl 1×binding buffer and measured by BD LSRFortessa flow cytometer. The data were analyzed using BD FACSDiva (Becton Dickinson, NJ, USA) software. Annexin V-positive and PI-negative cells were considered to be in the early apoptotic phase, Annexin V-negative and PI-positive cells were considered to be in the necrosis phase, cells having positive staining for both Annexin-V and PI were considered to undergo late apoptosis and cells negative for Annexin V and PI were considered to be live cells.

### Quantification and Statistical Analysis

Xenograft data were analyzed using parametric statistics as described in the legends. Survival curves were analyzed with log-rank statistics. In vitro experiments were analyzed using parametric statistics (ANOVA global test with Dunnett's/Sidak multiple comparison test as post-hoc tests as applicable and described in legend) and presented as the mean±SEM. All real time PCR's are relative semi quantitative RT-PCR's (hereby referred to as qRT-PCR) and are a combined quantitation of a minimum of 3 independent biological trials assayed in triplicate with biological replicates represented as individual scatter dots in the graphs or as indicated in legends. In all cases, statistical significance was set at a threshold of p<0.05. All statistical analyses were conducted with GraphPad Prism Software Ver. 9.0 and specific statistical test information described in figure legends.

### Results

### BMP2 and -9 Suppress Anchorage-Independent Survival and Promote Anoikis.

BMP2 was examined alongside BMP9 in a panel of OC cell lines. Cell lines representing a spectrum of OCs, including PA1 (ovarian teratocarcinoma), OVCA420 (serous adenocarcinoma), OVCAR3 (carcinoma high-grade serous), SKOV3 (carcinoma non-serous), and OVCA433 (serous adenocarcinoma) were grown under anchorage-independent suspension culture conditions. Treatment with BMP2 and BMP9 significantly decreased the live-to-dead cell ratio in spheroids (1.8-4.25 times decrease; FIG. 1(A)) with an increase in the percentage of dead cells (FIG. 15(A)). Spheroids treated with either BMP2 or BMP9also exhibited reduced 3D invasion capabilities (55%-67% reduction; FIG. 15(B)). As expected, both BMP2 and BMP9 did not alter cell growth for 3 days (FIG. 15C). These data indicate that both BMP2 and BMP9 promote anoikis and diminished spheroid invasion in a spectrum of OC lines.

### BMP9 Suppresses Metastatic Growth in the Peritoneal Cavity.

The effect of administering recombinant human (rh)BMP9 on peritoneal tumor growth and metastasis in vivo was evaluated. Delivery of BMP9 was used to mimic a potential therapeutic regimen. Overall toxicity of administering BMP9 i.p. daily, examined by body weight and kidney and liver function tests, revealed no notable toxicity for up to 3 weeks (FIGS. 15D and 15E).

The effect of BMP9 on in vivo tumor growth was tested by injecting PA1 cells with either vehicle or BMP9 into the peritoneal cavity of NOD-SCID mice, followed by daily BMP9 or vehicle administration. By using bioluminescence imaging (BLI), a significant reduction in overall i.p. tumor burden over time was observed in mice receiving BMP9 compared with mice receiving vehicle (FIGS. 1B and 1C). Mice euthanized upon morbidity at the end of a 7-week study for PA1 cells confirmed extensive tumor burden in the omentum (FIG. 1D) in the vehicle-treated group. In contrast, rhBMP9-treated mice had significantly lower omental tumor burden (FIG. 1D). Similar results were observed with a second cell line, SKOV3 (FIGS. 1E and IF). BMP9 treatment led to significantly lower intraperitoneal tumor growth as demonstrated by BLI in the abdomen by day 16 in the vehicle group (FIGS. 1E and IF). BMP9 administration also prolonged survival in mice compared with the vehicle group (FIG. 1G). Whereas all vehicle mice succumbed to disease between days 17 and 21 (FIG. 1G), BMP9-treated mice survived significantly longer, for between 30 and 40 days (FIG. 1G). Additionally, whereas all vehicle-treated mice had some ascites that was not measurable accurately, none of the rhBMP9-treated mice had any detectable ascites.

Histological comparison of tumors from both cell lines (PA1 and SKOV3) revealed tumor cells in large nodules in the omentum of the vehicle-treated group. In contrast, omental tumors from BMP9-treated mice consisted of significantly fewer infiltrated tumor cells (FIGS. 1H, 1I, and 15F). Apoptosis analysis by TUNEL staining revealed an increase in TUNEL-positive cells in tumors from BMP9-treated mice compared with vehicle-treated mice in both PA1-luc-GFP and SKOV3-luc-GFP groups (FIGS. 1H and 1I; a factor of two increase in PA1 and 143 in SKOV3). The increases in apoptosis and necrotic lesions (FIG. 15G) found in tumors from BMP9-treated mice was noted widely. ELISA analysis confirmed elevated BMP9 in the plasma from mice, verifying their presence in circulation (FIG. 15H). Thus, administration of (rh)BMP9 along with i.p.-injected tumor cells in vivo, which mimics the shedding of tumor cells into the peritoneal cavity during metastasis, suppresses intraperitoneal tumor spread and growth and prolongs survival of mice in two OC i.p.-xenograft models.

### SOX2 is a Repressed Transcriptional Target of BMP2, -4, and -9, but Not BMP 10, in Cancer.

To identify critical factors regulating anoikis in response to BMP, the transcription profile of 48,226 genes in PA1 cells cultured under anchorage-independent growth conditions treated with either BMP9 or vehicle control (FIG. 2A and FIG. 16A) were compared. Five-hundred and forty-three (543) differentially expressed genes using a 2-fold change criterion were found (p<0.05, GEO: GSE185924), which were further divided into upregulated (n=333) and downregulated genes (n=210) in response to BMP9 (FIG. 16A and Table 4). REACTOME analysis identified 18 pathways significantly altered, including BMP and TGF-β signaling, and transcriptional regulation of pluripotency-associated genes (FIG. 16B and Table 4). Notably, examination of the 30 top altered genes (15-up and 15-down) revealed SOX2, IGFBP5, and HTRID as the most repressed genes in BMP9-treated cells (12.37-to 20-fold change in gene expression; FIG. 2B). Additionally, among the top 30 altered genes, 28 were linked to SOX2 through PubMed searches (Card et al., 2008; Wang et al., 2015; Lach-mann et al., 2010; Si et al., 2019; Lim et al., 2007; Bani-Ya-ghoub et al., 2006; Ehlers and Todd, 2017; Kelberman et al., 2006; Seki, 2018; Acanda de la Rocha et al., 2016) and the GENECARD human gene database.

In OC cell lines, SOX2 expression at baseline is variable, with PA1 cells expressing the highest relative mRNA and protein level of SOX2, followed by OVCAR3 and SKOV3 (FIG. 2C). Using this panel as a guide, SOX2 downregulation were validated by BMP9 via qRT-PCR in anchorage-independent conditions (FIG. 16C). BMP9 did not have any significant effect on two other developmental transcription factors, OCT4 or NANOG (FIG. 16D). Reduction in SOX2 expression by BMP9 was significant in several cell lines even under attached growth conditions, including in OVCAR3 and SKOV3, which express detectable RNA and protein levels of SOX2 (FIGS. 2C and 2D). Furthermore, downregulation of SOX2 was mediated by two additional BMP ligands, BMP2 and BMP4 (FIG. 2E), with decreases at the protein (FIG. 2F) and RNA levels in both PA1 and OVCAR3 cells (FIG. 2G). BMP4 also promotes anoikis, as treatment with BMP4 significantly decreased the live-to-dead cell ratio in PA1 cells under anchorage independence (1.93; FIG. 16E). However, BMP10, which exhibits the highest sequence homology to BMP9 (Tillet and Bailly, 2014), did not alter SOX2 (FIG. 2E), or anoikis (FIG. 16F).

BMP9-mediated SOX2 repression also occurred in xenograft tumors, as IHC analysis of SOX2 and qRT-PCR analysis revealed an overall reduction in SOX2 levels in tumors from MP9-treated mice compared with vehicle control-treated mice (FIGS. 2H and 18G). Importantly, patient ascites-derived tumor cells EOC15 and AF68 express SOX2 under anchorage-independent conditions, which was downregulated by BMP9 treatment as well (FIG. 2I). In addition to OC, several other cancer types are known to express SOX2, including lung (Ochieng et al., 2014), pancreatic neuroendocrine, and bronchial carcinoid tumor (Akiyama et al., 2016). BMP2 and BMP9 treatment downregulated SOX2 expression in A549 (lung cancer), BON-1 (P-NET), and H727 (bronchial carcinoid tumor) cells as well (FIG. 2J).

OVCAR4, OVCA420, and OVCA433 cells (low SOX2 expression) significantly upregulate SOX2 expression under anchorage independence (FIG. 2K). These increases in SOX2 were effectively suppressed by both BMP2 and BMP9 (FIG. 2L). Changes in SOX2 under anchorage independence were not restricted to low-SOX2 cell lines but were also measurable in cell lines with higher baseline levels, including OVCAR3 and PA1 (FIGS. 17A and 17B), which were also suppressed by BMPs (FIG. 16C). All together, these results indicate that BMP2, -4, and -9 can downregulate SOX2 in multiple cancer types either when endogenous levels are high or when SOX2 expression is induced in response to anchorage-independent growth or during in vivo tumor progression.

### Rapid Transcriptional Downregulation of SOX2 is Sufficient for Anoikis.

Kinetics and dose-response studies reveal dose-dependent repression of SOX2 by both BMP2 and BMP9, with BMP9 being pronounced at lower doses compared with BMP2 (FIG. 3A). SOX2 protein repression began within 6 h post-treatment and by 2 h at the mRNA level in both PA1 (FIG. 3B) and OVCAR3 cells (FIG. 3C). Both BMP2 and BMP9 significantly reduce luciferase activity of a 1-kb SOX2 promoter reporter (Yeh et al., 2018) (FIG. 3D). Expressing SOX2 from a heterologous promoter (CMV) prevented BMP2-and BMP9-mediated decreases in SOX2 in SKOV3 cells (FIG. 3E). Similar overexpression of SOX2 from a different heterologous promoter (EF1a) in PA1 cells that express high levels of endogenous SOX2, was able to suppress the decrease of SOX2 by BMP2 and BMP9 (FIG. 3F), accounting for both the endogenous and the heterologous EF1a-driven SOX2. Overexpression of SOX2 from a heterologous promoter resulted in suppressing anoikis (increased anchorage-independent survival) and generated compact spheroids compared with control cells (FIG. 3G; CMV-SOX2). BMPs, however, had no significant effect on anoikis in cells overexpressing SOX2 from the CMV promoter (CMV-SOX2; FIG. 3H) compared with that in control cells (SKOV3-CMV-control; FIG. 3H). These data demonstrate that SOX2 is necessary for survival under anchorage independence by inhibiting anoikis and is a major transcriptionally repressed target of BMP for anoikis.

### Patient Ascites are Low in BMP9 but High in TGF-β, Which Upregulates SOX2 and Promotes Anchorage-Independent Survival by Suppressing Anoikis.

To evaluate the levels of BMPs and other TGF-β members, particularly (TGF-β1/2), in OC patient ascites, an environment bearing tumor cells under anchorage independence, ligand-specific ELISAs were used here. BMP9 could not be detected in patient ascites irrespective of disease stage (FIG. 4A). In contrast, significantly higher levels of TGF-β1 (3800-52,348 pg/mL) and TGF-β2 (64-4,259 pg/mL) were present, with TGF-β1 being an order magnitude higher than TGF-β2 (FIG. 4A). On the basis of these observations, the effect of TGF-β1 on SOX2 was tested. In contrast to BMP2 and -9, TGF-β1 increased SOX2 protein and mRNA expression under both attached (FIGS. 4B-4D) and anchorage-independent conditions (FIG. 18A). Activin, another TGF-β1 member, also increased SOX2 levels, like TGF-β1 (FIG. 4B). In a corollary fashion, live-dead analysis of anchorage-independent spheroids treated with TGF-β1 increased the live/dead ratio in OC cells (PA1, OVCA420, and OVCAR3; FIG. 4E), with spheroids treated with activin A demonstrating a similar trend in reduction of cell death under anchorage independence (FIG. 18B). Luciferase activity of the 1-kb SOX2 promoter reporter construct (Yeh et al., 2018) was increased in response to TGF-β1 treatment (FIG. 4F) and activin A (FIG. 18C) as well.

Since BMP9 and TGF-β1 have opposing effects on SOX2, and TGF-β1 is present at high levels in the ascites of patients (FIG. 4A), whether TGF-β1 or activin A would over-ride the effects of BMP9 on SOX2 repression was next evaluated. Equimolar amounts of BMP9 or TGF-β1 either decreased or increased SOX2 respectively, whereas the combination treatment led to a 70% reduction in SOX2 (FIG. 4G). Similar lowering of SOX2 was observed when activin A and BMP9 were combined (FIG. 18D). These findings on the differential effects of BMP and TGF-β on SOX2 and anoikis (FIGS. 3 and 4) point to SOX2 as an important node determining anchorage-independent survival in response to TGF-β ligands and implicate BMP9 as a feasible way to override the effects of TGF-β on SOX2.

### SOX2 Levels are Differentially Regulated by ALK2, ALK3, and ALK5.

The differential effects of TGF-β ligands and the difference in the extents of SOX2 repression between BMP ligand isoforms (BMP9>>BMP2; FIGS. 3 and 4B), prompted further study to delineate receptor and SMAD signaling involvement down-stream of these ligands. A panel of small-molecule inhibitors was used to the different type I (ALK) receptors; Dorsomorphin (DM, ALK2/3/6 [Hao et al., 2008]); SB431542 (ALK4/5/7 [Inman et al., 2002]); ML347, (ALK1/2 [Engers et al., 2013]), and LDN193189 (ALK2/3 [Boergermann et al., 2010]). Whereas BMP9 repressed SOX2 both at the protein and mRNA level in vehicle-control cells (FIG. 5A in PA1; FIG. 19A for OVCAR3), inhibiting ALK2/3/6 resulted in 34.3% recovery in SOX2 levels in the presence of BMP9 (FIG. 5A, DM lanes). Inhibiting ALK4/5/7 did not significantly alter the extent of SOX2 repression by BMP (FIG. 5A, SB lanes). Similarly, inhibiting ALK2/3/6 in BMP2-treated cells resulted in a 23% recovery in SOX2 repression (FIG. 5B, DM lanes, and FIG. 19B). Again, inhibiting ALK4/5/7 did not alter the extent of SOX2 repression by BMP2 (FIG. 5B, SB lanes). Phosphorylated SMAD1 (pSMAD1) in response to BMP2 and -9 was repressed by DM (ALK2,3,6 inhibition), with no effects observed upon co-treatment with SB431542 (ALK4/5/7 inhibition). Interestingly, inhibiting ALK1/2 with ML347 increased baseline SOX2 levels even in the absence of exogenous ligand (FIG. 5C, ML347 lane 4) and abrogated the ability of BMP9 to repress SOX2 at both protein and mRNA levels in PA1 (FIG. 5C, ML347+BMP9 compared with BMP9 lanes) and OVCAR3 cells (FIG. 19C). Compared with BMP9, ALK1/2 inhibition only partially prevented BMP2-mediated SOX2 repression (72% recovery; FIG. 5C, ML347+BMP2 compared with BMP2 lanes). BMP9-induced phosphorylation of SMAD1 was completely suppressed with ML347 (FIG. 5C, ML347 lanes, and FIG. 19C), with only partial (40%) suppression of BMP2-induced SMAD1 phosphorylation (FIG. 5C). Similarly, inhibition of ALK2,3 using LDN193189increased SOX2 protein levels at baseline even in the absence of exogenous ligand (33; FIG. 5C) and blocked both BMP2's and BMP9's ability to repress SOX2 (FIG. 5C). Full inhibition of BMP2- and BMP9-induced SMAD1/5 phosphorylation was also observed (FIG. 5C, LDN lanes, and FIG. 19C, OVCAR3 cells). All together, these data demonstrate a strong preference for ALK2 in BMP9-dependent SOX2 repression at both the protein and RNA levels and a combination of ALK2 and ALK3 in BMP2-dependent SOX2 repression.

To confirm specific roles of ALK2 and ALK3 receptors in SOX2 repression, constitutively active kinases ALK2 or ALK3 (HA-ALK2QD and HA-ALK3QD) (Imamura et al., 1997) were expressed in PA1 and OVCAR3 cells. Activating ALK2 kinase (ALK2QD) decreased SOX2 even in the absence of exogenous BMP ligand (69% reduction in PA1 and 90% in OVCAR3; FIGS. 5D and 5E). In the presence of ligand (BMP2, BMP9), ALK2QD-mediated SOX2 repression was further enhanced (FIGS. 5D and 5E). The effect of activating ALK3 (ALK3QD) was modest compared with ALK2QD and was cell line dependent. ALK3QD did not reduce SOX2 in the absence of exogenous ligand in PA1 cells but was able to reduce SOX2 levels by 65% in OVCAR3 cells in the absence of exogenous ligand (FIGS. 5D and 5E). The presence of ligand (BMP2, BMP9) only slightly enhanced SOX2 repression in both cell lines with ALK3QD (FIGS. 5D and 5E). These findings demonstrate a requirement for both ALK2 and ALK3, with ALK2 being critical for maximum SOX2 downregulation based on ligand-independent effects and enhancement of the effects of both BMP9 and BMP2.

Since both TGF-β1/2, and activin predominantly utilize ALK4/5/7 for phosphorylating SMAD2/3, the effect of blocking their kinase activity using SB431542 was evaluated. It was found that SB431542 suppressed TGF-β1-induced SOX2 increases (FIGS. 5F and 5G). These studies together implicate different ALK receptors: ALK2 and ALK3 in SOX2 repression and ALK4 and ALK5 in increasing SOX2 expression.

### SMAD1 and SMAD3 Differentially Regulate SOX2 and Occupy the SOX2 Promoter in Response to BMP9 and TGF-β, Respectively.

SMAD1 phosphorylation is a primary response to ALK2 and ALK3 kinases (Heldin and Moustakas, 2016) that regulate SOX2 levels downstream of BMP (FIG. 5). A direct role for SMAD1 in SOX2 repression was tested by using pooled shRNAs to SMAD1 (shSMAD1). Reducing SMAD1 significantly decreased the ability of BMP2 and BMP9 to reduce SOX2 levels compared with control shRNA cells (shNTC) by 30-44% (FIG. 6A). Since ALK5, downstream of TGF-β signaling (FIGS. 5F and 5G), primarily phosphorylates SMAD2/3 (Heldin and Moustakas, 2016), SMAD3 was silenced using pooled siRNAs (FIG. 6B). TGF-β increased SOX2 levels in control (siScr) cells (FIG. 6B) but was unable to increase SOX2 in siSMAD3 cells (FIG. 6B). Strikingly, siRNA to SMAD3 also lowered SOX2 levels at the baseline even in the absence of exogenous ligands (FIG. 6B), indicating direct roles for SMAD3 in SOX2 upregulation. A compensatory increase in pSMAD1 upon lowering of SMAD3 (FIG. 6B) correlated with lower SOX2 levels even in the presence of TGF-β1 (FIG. 6B).

In silico analysis revealed several SMAD1- and SMAD3-binding motifs (GG(C/A)GCC and GTCT/AGAC, respectively) within 2 kb of the transcriptional start site for SOX2 (TSS; FIG. 6C) (Martin-Malpartida et al., 2017). Chromatin immunoprecipitation (ChIP) was used to assess the binding of SMADs to these sites. The promoter contains four SMAD1 and two SMAD3 motifs. However, due to several “CG” clusters (CpG islands), primers were designed, flanking regions immediately outside the CpG islands with additional sites within 2 kb of the TSS (FIG. 6C). Primers flanking SMAD1-binding elements (p1, p2, p5, p6) and primers flanking SMAD3-binding elements (p1, p3, p4, and p5) were used, with p1 and p5 having both SMAD1- and SMAD3-binding elements, and p6 located closest to the TSS (FIG. 6C). BMP9 treatment led to a significant enrichment of SMAD1 binding at two sites: p1 and p6 (FIG. 6D). TGF-β1 treatment led to consistent SMAD3 enrichment at p1, p3, p4, and p5 (FIG. 6E). Due to the proximity of p6 to a SMAD3-binding element, SMAD3 enrichment was tested and found at p6 as well in response to TGF-β1 treatment (FIG. 6E). These sites were also tested for response to activin A and were similarly found to be occupied by SMAD3 (FIG. 20). These data together indicate enrichment of SMAD1 and SMAD3 to SOX2's promoter and upstream regions in response to BMP9, TGF-β1, and activin A, respectively, with one site occupied by both SMAD1 and SMAD3 and other uniquely occupied regions.

### Epigenetic Regulation of SOX2 is Mediated by SMAD-dependent Methylated Histone Occupancy and Promoter DNA Methylation.

Gene repression and activation by SMADs frequently require additional proteins and chromatin modification (Hill, 2016). Hence, whether protein degradation was required for BMP9-induced SOX2 repression was evaluated. No effect of MG132, an inhibitor of proteasomal degradation, was found on BMP's ability to repress SOX2 (FIG. 21A). However, histone H3K27me3 was significantly enriched on the SOX2 promoter in response to BMP9 treatment (FIG. 6F). This enrichment was SMAD1-signaling dependent, as H3K27me3 occupancy was significantly reduced in the presence of LDN193189 (ALK/SMAD1 inhibitor; FIG. 6F). Notably, lowering global histone H3K27me3 in PA1, SKOV3, and OVCAR3 cells by using GSK126, an inhibitor to EZH2, (McCabe et al., 2012) (FIG. 21B) abrogated SOX2 repression by BMP9 in multiple cell lines (FIGS. 6G and 21C). These data demonstrate a role for H3K27me3 in transcriptional repression of SOX2 downstream of BMP9 signaling.

TGF-β1 treatment, conversely, led to enrichment of H3K4me3 at multiple SMAD3 motifs (FIG. 6H; p1, p3, p4, p5, p6). This enrichment was SMAD3 dependent, as H3K4me3 enrichment was abrogated by SB431542 treatment (FIG. 6H). These regions are consistent with ENCODE analysis in HeLa and A549 cell lines (FIG. 6C) that identified highest H3K27me3 peaks at p5 and p6, the same regions where SMAD1 enrichment in response to BMP9 (FIG. 6D); p1 exhibited a high peak of H3K4me3, the same SMAD3-occupied region in response to TGF-β1. This suggests that SMAD1 signaling and occupancy lead to an increase in histone H3K27me3, and conversely, SMAD3 signaling and occupancy lead to an increase in histone H3K4me3 at SOX2's promoter and regulatory regions.

Due to the presence of CpG islands within 10 bp of the p6 primer sites, (FIG. 6C), DNA methylation in response to BMP9 was evaluated. Methylation-specific qPCR in response to BMP9 revealed a 2.53 increase in SOX2 promoter methylation compared with control cells (FIG. 6I). The DNA methyltransferase (DNMT) inhibitor 50-azacytidine (50-Aza) (Yang et al., 2017), abrogated BMP9-induced SOX2 promoter methylation (FIG. 6I) and in parallel suppressed SOX2 mRNA downregulation by BMP9(FIG. 6J). Interestingly, a minimal effect on SOX2 promoter methylation in PA1 cells treated with 50-Aza alone was noted (FIG. 6I). However, compared with PA1 cells, which express high SOX2 (FIG. 2C), cells with lower baseline levels of SOX2 such as SKOV3 (FIG. 2C) suppressed SOX2 promoter methylation significantly upon 50-Aza treatment (FIG. 21D), with a concomitant increase in SOX2 mRNA (FIG. 21E). 50-Aza also repressed BMP9-induced SOX2 transcriptional repression in SKOV3 cells in a similar manner to that in PA1 cells (FIG. 6K). These findings confirm robust regulation of the SOX2 locus by methylation in different cell lines and indicate that DNA methylation along with SMAD1-dependent H3K27me3 recruitment can drive SOX2 repression.

SOX2 repression leads to genome-wide changes in key transcriptional factors and cell death pathways under anchorage independence

High SOX2 is associated with a poor prognosis for OC patients (Zhang et al., 2012), and reducing SOX2 expression transiently using pooled siRNAs (siSOX2; FIG. 7A) or alternatively stably using shRNA (shSOX2; FIG. 7B) resulted in increased cell death under anchorage independence (anoikis) compared with control cells (FIGS. 7A and 7B). In both siSOX2 and shSOX2 cells, spheroids appeared disaggregated and less compact compared with their respective controls (FIGS. 7A and 7B). The requirement of SOX2 for anchorage-independent survival led to exploring genes and pathways impacted by SOX2 specifically under anchorage independence. Genome-wide gene expression profiles of siSOX2 and siNTC (non-targeting control) PA1 cells were compared using RNA-sequencing from cells under anchorage independence. This analysis described here revealed 59differentially expressed genes (DEGs) between siNTC and siSOX2 cells (p % 0.05; FIG. 7C and Table 5), with 24 of these downregulated and 35 upregulated in siSOX2 cells compared with control (FIG. 7C and Table 5). Of the total 59 DEGs, 21 of them including SOX2 also changed their expression levels in response to BMP9 from the microarray analysis described herein (FIGS. 7D and 24A). A closer analysis of the 20 genes for SOX2-binding motifs within 1 kb of the transcription start sites using LASAGNA-Search, revealed that 17 of 20 of the common DEGs presented one or more SOX2-binding motifs (FIG. 22B). Downregulated DEGs had previously been implicated in processes relevant to cell adhesion (POSTN [Soikkeli et al., 2010]) and metastasis (TRIM22 [Ji et al., 2021]). Gene set enrichment analysis of the differentially expressed genes from the RNA-seq data revealed enrichment of eight Hallmark gene sets based on a FDR value<25% and included “apoptosis,” “TGF-β signaling,” and “epithelial-mesenchymal transition” in siSOX2 cells (FIG. 7E) and, interestingly, “interferon alpha response” and “interferon gamma response” in the control siNTC cells (FIG. 7F). Upregulation of several genes from the apoptosis pathway, including BMF, BCL2L11, and BID (FIG. 7E) were confirmed to be upregulated in siSOX2 cells under anchorage independence (FIG. 22C). Genes from the TGF-β signaling pathway were also analyzed and ACVRI (ALK2) was identified as one of the upregulated genes upon silencing SOX2 (FIG. 22C). Additional validated genes from the RNA seq analysis included TRIM22, CD47, and CD74 genes from the interferon alpha and gamma response pathways in control (siNTC) cells (FIGS. 22C and 22D). It was found TRIM22 to be downregulated in siSOX2 cells (FIG. 22C), whereas CD47 and CD74 were up-regulated in siSOX2 cells to different extents (FIG. 22D). Taken together, these findings establish a role for SOX2 silencing in promoting apoptosis under anchorage independence with alterations to key transcriptional and epigenetic regulators and adhesion molecules for tumor cell survival.

BMP9 Suppressed OVCA cell Survival Under Anchorage Independence.

HEY parental and HEYT30 were treated with increasing doses of BMP9 (1, 5, 10 nM) for 24, 48, and 72 hours. BMP9 significantly decreased the percentage live cells by 50-65% in HEY parental (FIG. 8A). However, BMP9 had a lesser effect in HEYT30 with about 35% reduction at 1 and 5 nM and 63% with 10 nM (FIG. 8B).

The kinetics and dose effect of BMP9 was also tested in the cisplatin chemotherapy pairs: cisplatin-sensitive A2780ip2 and cisplatin-resistant A2780CP (FIG. 9). The cisplatin-resistant A2780CP were found to be significantly more responsive to BMP9treatment compared to cisplatin-sensitive A2780ip2 cells (FIG. 9A, B). The decrease in percentage live cells was by 30% in A2780ip2 (FIG. 9A) as opposed to 25-55% in A2780CP (FIG. 9B). These findings suggest that p53 genetic mutation status and cisplatin chemoresistant cells are sensitive to BMP9 treatment compared to their parental controls.

BMP9 enhances chemosensitivity to Paclitaxel in resistant ovarian cancer cells.

The 3D live dead analysis in FIG. 8 revealed that HEYT30 cells had a lower response to BMP9 treatment compared to HEY parental cells, so whether combination of BMP9 and paclitaxel could have therapeutic benefit in HEYT30 resistant cells was investigated. Paclitaxel-resistant HEYT30 cells were treated with increasing doses of paclitaxel (0 nM-258 nM) and either simultaneously exposed or were pretreated for 24 hours to 1 nM, 5 nM, or 10 nM BMP9. Simultaneous BMP9 treatment reduced cell viability in HEYT30 by lowering the IC₅₀ concentration of paclitaxel in a dose-dependent manner (FIG. 10A). Untreated control HEYT30 cells had a paclitaxel IC₅₀ concentration of 106.7 nM, 1 nM BMP9 decreased the IC₅₀ concentration to 91.7 nM, 5 nM BMP9 decreased the IC₅₀ concentration to 89 nM, and 10 nM BMP9 decreased the IC₅₀ concentration to 73 nM (FIG. 10A). Strikingly, 24-hour pretreatment to BMP9 further significantly reduced cell viability and lowered the IC₅₀ concentration of paclitaxel in a dose-dependent manner in HEYT30 (FIG. 10B). Untreated control HEYT30 cells had a paclitaxel IC₅₀ concentration of 113.2 nM, 1 nM BMP9 decreased the IC₅₀ concentration to 90.8 nM, 5 nM BMP9 decreased the IC₅₀ concentration to 22.6 nM, and 10 nM BMP9 decreased the IC₅₀ concentration to 4.1 nM (FIG. 10B).

Twenty-four hour pretreatment with BMP9 significantly decreased paclitaxel IC₅₀, and the effect of the 24-hour BMP9 pretreatment on paclitaxel chemosensitivity under 3D conditions was investigated (FIG. 11). Paclitaxel-resistant HEYT30 cells treated with increasing doses of Paclitaxel (1 nM-512 nM) in the presence of 24-hour pretreatment with 5 nM and 10 nM BMP9 had a lowered IC₅₀ concentration compared to control cells. This study showed an increase in baseline paclitaxel IC₅₀ concentration of 312 nM in untreated control HEYT30 cells under 3D conditions (FIG. 11). 5 nM BMP9 decreased the IC₅₀ concentration to 285.5 nM, and 10 nM BMP9 decreased the IC₅₀ concentration to 210.9 nM (FIG. 11).

Co-treatment with Paclitaxel and BMP9 Decreases Colony Formation in Ovarian Cancer Cells.

The colony formation effect of co-treatment of BMP9 and paclitaxel in HEYT30 cells was further assessed. Clonogenic assay to assess the effect of co-treatment of BMP9 and paclitaxel in HEYT30 cells revealed that combination of 10 nM BMP9 and 80 nM paclitaxel significantly diminished the colony formation ability of HEY-T30 cells compared to individual treatments (FIG. 12), suggesting that combinatorial treatment of BMP9 and paclitaxel effectively exacerbate cell viability in HEY-T30 cells.

### Combination of BMP9 and Paclitaxel Shows Enhanced Cytotoxicity in Resistant Ovarian Cancer Cells.

To examine the effect of combinatorial treatment of BMP9 and paclitaxel in HEY-T30 cells, 3 doses were investigated (below and above paclitaxel IC50 in HEYT30). Combination of the 3 doses of paclitaxel (64, 128, 256 nM) and BMP9 (1, 5, 10 nM) under 2D conditions, and paclitaxel (128, 256, 512 nM) and BMP9 (5, 10 nM) under 3D condition, alone and in combination for 48 hours, revealed that combinatorial treatment significantly increased cytotoxicity measured by a reduction in cell viability, as opposed to individual treatment (FIG. 13A, B). A concentration of 10 nM BMP9 enhanced cytotoxicity visibly at concentrations 64, 128, and 256 nM in HEYT30 cells (FIG. 13C). Staining HEY-T30 cells with propidium iodide and Annexin V apoptosis detection kit to determine the effect of combinatorial treatment on cell apoptosis, revealed that combination treatment resulted in 13.3% apoptosis in cells, which is higher than 12.8% caused by 300 nM paclitaxel or 6.8% caused by 10 nM BMP9 alone (FIG. 14).

### LIST OF REFERENCES

1. Acanda de la Rocha et al. Analysis of SOX2-regulated transcriptome in glioma stem cells. PLoS One. 2016, 11:00163155
2. Ahmed N. et al. Getting to know ovarian cancer ascites: opportunities for targeted therapy-based translational research. Front. Oncol. 2013; 3: 256.
3. Akiyama T. et al., Expression of sex determining region Y-box 2 and pancreatic and duodenal homeobox 1 in pancreatic neuroendocrine tumors. Pancreas. 2016; 45:522-527.
4. Alonso M.M. et al., Genetic and epigenetic modifications of Sox2 contribute to the invasive phenotype of malignant gliomas. PLoS One. 2011; 6: e26740.
5. Bååth M. et al. SOX2 is a promising predictor of relapse and death in advanced stage high-grade serous ovarian cancer patients with residual disease after debulking surgery. Mol. Cell Oncol. 2020; 7: 1805094.
6. Bani-Yaghoub M. et al. Role of Sox2 in the development of the mouse neocortex. Dev. Biol. 2006; 295: 52-66.
7. Bareiss P.M. et al. SOX2 expression associates with stem cell state in human ovarian carcinoma. Cancer Res. 2013; 73: 5544-5555.
8. Belotte J. et al. Sox2 gene amplification significantly impacts overall survival in serous epithelial ovarian cancer. Reprod. Sci. 2015; 22: 38-46.
9. Boergermann J.H. et al. Dorsomorphin and LDN-193189 inhibit BMP-mediated Smad, p38 and Akt signalling in C2C12 cells. Int. J. Biochem. Cell Biol. 2010; 42: 1802-1807.
10. Cai Q. et al. Anoikis resistance is a critical feature of highly aggressive ovarian cancer cells. Oncogene. 2015; 34: 3315-3324.
11. Canali S. et al. Activin B induces noncanonical SMAD1/5/8 signaling via BMP type I receptors in hepatocytes: evidence for a role in hepcidin induction by inflammation in male mice. Endocrinology. 2016; 157: 1146-1162.
12. Candia A.F. et al. Cellular interpretation of multiple TGF-beta signals: intracellular antagonism between activin/BVg1 and BMP-2/4 signaling mediated by Smads. Development. 1997; 124: 4467-4480.
13. Cao Z. et al. Anoikis and EMT: lethal “liaisons” during cancer progression. Crit. Rev. Oncog. 2016; 21: 155-168.
14. Card D.A.G. et al. Oct4/Sox2-regulated miR-302 targets cyclin D1 in human embryonic stem cells. Mol. Cell Biol. 2008; 28: 6426-6438.
15. David C.J. et al. Contextual determinants of TGFβ action in development, immunity and cancer. Nat. Rev. Mol. Cell Biol. 2018; 19: 419-435.
16. David L. et al. Identification of BMP9 and BMP10 as functional activators of the orphan activin receptor-like kinase 1 (ALK1) in endothelial cells Blood. 2007; 109. 1953-1961.
17. Dobin A. et al. STAR: ultrafast universal RNA-seq aligner. Bioinformatics. 2013; 29: 15-21.
18. Doré J.J. et al. Heteromeric and homomeric transforming growth factor-beta receptors show distinct signaling and endocytic responses in epithelial cells. J. Biol. Chem. 1998; 273:31770-31777.
19. Ehlers M.R. et al. Genesis and maintenance of attentional biases: the role of the locos coeruleus-noradrenaline system. Neural Plast. 2017; 2017:6817349.
20. Ehrlich M. et al. Oligomeric interactions of TGF-beta and BMP receptors. FEBS Lett. 2012; 586:1885-1896.
21. Engers D.W. et al. Synthesis and structure-activity relationships of a novel and selective bone morphogenetic protein receptor (BMP) inhibitor derived from the pyrazolo[1.5-a]pyrimidine scaffold of dorsomorphin: the discovery of ML347 as an ALK2 versus ALK3 selective MLPCN probe. Bioorg. Med. Chem. Lett. 2013, 23:3248-3252.
22. Frisch S.M. Disruption of epithelial cell-matrix interactions induces apoptosis. J. Cell Biol. 1994; 124:619-626.
23. Frisch S.M. et al. Mechanisms that link the oncogenic epithelial-mesenchymal transition to suppression of anoikis. J. Cell Sci. 2013; 126: 21-29.
24. Hao J. et al. Dorsomorphin, a selective small molecule inhibitor of BMP signaling, promotes cardiomyogenesis in embryonic stem cells. PLoS One. 2008; 3: e2904.
25. Heldin C.-H. et al. Signaling receptors for TGF-β family members. Cold Spring Harb. Perspect. Biol. 2016; 8: a022053.
26. Hellner K. et al. Premalignant SOX2 overexpression in the fallopian tubes of ovarian cancer patients: discovery and validation studies EBioMedicine. 2016, 10:137-149
27. Hill C.S. Transcriptional control by the SMADs. Cold Spring Harb. Perspect. Biol. 2016, 8: a022079.
28. Holtzhausen A.et al. Novel bone morphogenetic protein signaling through Smad2 and Smad3 to regulate cancer progression and development. FASEB.J. 2014; 28: 1248-1267.
29. Huang H. et al. Clinical significance of ascites in epithelial ovarian cancer. Neoplasma. 2013; 60: 546-552.
30. Ikushima H. et al. Autocrine TGF-beta signaling maintains tumorigenicity of glioma-initiating cells through Sry-related HMG-box factors Cell Stem Cell. 2009; 5: 666
31. Imamura T. et al. Smad6 inhibits signalling by the TGF-β superfamily. Nature. 1997; 389: 622-626.
32. Inman G.J. et al. SB-431542 is a potent and specific inhibitor of transforming growth factor-beta superfamily type I activin receptor-like kinase (ALK) receptors ALK4, ALK5, and ALK7. Mol. Pharmacol. 2002; 62: 65-74.
33. Ji J. et al. TRIM22 activates NF-κB signaling in glioblastoma by accelerating the degradation of IκBα. Cell Death Differ. 2021, 28: 367-381.
34. Kelberman D.et al. Mutations within Sox2/SOX2 are associated with abnormalities in the hypothalamusuitary-gonadal axis in mice and humans. J. Clin. Invest. 2006; 116: 2442-2455.
35. Kim Y.S. et al. Context-dependent activation of SIRT3 is necessary for anchorage-independent survival and metastasis of ovarian cancer cells. Oncogene. 2020; 39: 1619-1633.
36. Lachmann A. et al. ChEA: transcription factor regulation inferred from integrating genome-wide ChIP-X experiments. Bioinformatics. 2010; 26: 2438-2444
37. Lane D. et al. Inflammation-regulating factors in ascites as predictive biomarkers of drug resistance and progression-free survival in serous epithelial ovarian cancers. BMC Cancer. 2015; 15: 492.
38. Lim L.S. et al. Zic3 is required for maintenance of pluripotency in embryonic stem cells. Mol. Biol. Cell. 2007; 18: 1348-1358.
39. Liu I.M. et al. TGFbeta-stimulated Smad1/5 phosphorylation requires the ALK5 L45 loop and mediates the pro-migratory TGFbeta switch. EMBO J. 2009; 28: 88-98.
40. Liu Y. et al. Modulation of circulating protein biomarkers in cancer patients receiving bevacizumab and the anti-endoglin antibody, TRC105. Mol. Cancer Ther. 2018, 17: 2248-2256.
41. Love M.I. et al. Moderated estimation of fold change and dispersion for RNA-seq data with DESeq2. Genome Biol. 2014; 15: 550.
42. Luo J. et al. TGFβ/BMP type 1 receptors ALK1 and ALK2 are essential for BMP9-induced osteogenic signaling in mesenchymal stem cells. J. Biol. Chem. 2010; 285: 29588-29598.
43. Martin-Malpartida P. et al. Structural basis for genome wide recognition of 5-bp GC motifs by SMAD transcription factors Nat. Commun. 2017, 8: 2070.
44. Martinez-Hackert E. et al. Receptor binding competition: a paradigm for regulating TGF-beta family action. Cytokine Growth Factor Rev. 2021; 57: 39-54.
45. McCabe M.T. et al. EZH2 inhibition as a therapeutic strategy for lymphoma with EZH2-activating mutations. Nature. 2012; 492: 108-112.
46. Medeiros R.B. et al. Novel sequential ChIP and simplified basic ChIP protocols for promoter co-occupancy and target gene identification in human embryonic stem cells. BMC Biotechnol. 2009; 9: 59.
47. Miyazono K. et al. BMP receptor signaling: transcriptional targets, regulation of signals, and signaling cross-talk. Cytokine Growth Factor Rev. 2005, 16: 251-263.
48. Monavarian M. et al. Emerging perspectives on growth factor metabolic relationships in the ovarian cancer ascites environment. Semin. Cancer Biol. 2022.
49. Naber H.P.H. et al. Spheroid assay to measure TGF-β-induced invasion. J. Vis. Exp. 2011; 3337 .
50. Neckmann U. et al. GREM1 is associated with metastasis and predicts poor prognosis in ER-negative breast cancer patients. Cell Commun. Signal. 2019; 17: 140.
51. Ochieng J.K. et al. Sox2 regulates the emergence of lung basal cells by directly activating the transcription of Trp63. Am. J. Respir. Cell Mol. Biol. 2014; 51: 311-322.
52. Olsen O.E. et al. Bone morphogenetic protein-9 suppresses growth of myeloma cells by signaling through ALK2 but is inhibited by endoglin. Blood Cancer J. 2014; 4: e196.
53. Patro R. et al. Salmon provides fast and bias-aware quantification of transcript expression. Nat. Methods. 2017; 14: 417-419.
54. Peng J. et al. The BMP signaling pathway leads to enhanced proliferation in serous ovarian cancer-A potential therapeutic target. Mol. Carcinog. 2016; 55: 335-345.
55. Pham D.L. et al. SOX2 expression and prognostic significance in ovarian carcinoma. Int. J. Gynecol. Pathol. 2013; 32: 358-367.
56. Ramachandran A. et al. TGF-β uses a novel mode of receptor activation to phosphorylate SMAD1/5 and induce epithelial-to-mesenchymal transition. Elife. 2018; 7: e31756.
57. Ren W. et al. BMP9 inhibits the bone metastasis of breast cancer cells by downregulating CCN2 (connective tissue growth factor, CTGF) expression. Mol. Biol. Rep. 2014; 41: 1373-1383.
58. Ross S. et al. How the Smads regulate transcription. Int. J. Biochem. Cell Biol. 2008; 40: 383-408.
59. Scharpfenecker M. et al. BMP-9 signals via ALK1 and inhibits bFGF-induced endothelial cell proliferation and VEGF-stimulated angiogenesis. J. Cell Sci. 2007; 120: 964-972
60. Seki Y. et al. PRDM14 is a unique epigenetic regulator stabilizing transcriptional networks for pluripotency. Front. Cell Dev. Biol. 2018, 6-12.
61. Sholl L.M. et al. Sox2 protein expression is an independent poor prognostic indicator in stage I lung adenocarcinoma. Am. J. Surg. Pathol. 2010; 34: 1193-1198.
62. Si J. et al. Shisa3 brakes resistance to EGFR-TKIs in lung adenocarcinoma by suppressing cancer stem cell properties. J. Exp. Clin. Cancer Res. 2019; 38: 481.
63. Sinclair A.H. et al. A gene from the human sex-determining region encodes a protein with homology to a conserved DNA-binding motif. Nature. 1990; 346: 240-244.
64. Singh A. et al. The Yin and Yang of bone morphogenetic proteins in cancer. Cytokine Growth Factor Rev. 2010; 21: 299-313.
65. Soikkeli J. et al. Metastatic outgrowth encompasses COL-I, FN1, and POSTN op-regulation and assembly to fibrillar networks regulating cell adhesion, migration, and growth. Am. J. Pathol. 2010; 177: 387-403.
66. Soufi A. et al. Facilitators and impediments of the pluripotency reprogramming factors' initial engagement with the genome. Cell. 2012; 151: 994-1004.
67. Stirling D.R. et al. CellProfiler 4: improvements in speed, utility and usability. BMC Bioinf. 2021; 22: 433.
68. Sun C. et al. Sox2 expression predicts poor survival of hepatocellular carcinoma patients and it promotes liver cancer cell invasion by activating Slug. Med. Oncol. 2013; 30: 503.
69. Tao J.J. et al. First-in-Homan phase I study of the activin A inhibitor, STM 434, in patients with granulosa cell ovarian cancer and other advanced solid tumors. Clin. Cancer Res. 2019; 25: 5458-5465.
70. Tillet E. et al. Emerging roles of BMP9 and BMP10 in hereditary hemorrhagic telangiectasia. Front. Genet. 2014; 5: 456.
71. Torchiaro E. et al. Peritoneal and hematogenous metastases of ovarian cancer cells are both controlled by the p90RSK through a self-reinforcing cell autonomous mechanism. Oncotarget. 2016, 7: 712-728.
72. Varadaraj A. et al. Epigenetic regulation of GDF2 suppresses anoikis in ovarian and breast epithelia. Neoplasia. 2015; 17: 826-838.
73. Viallard C. et al. BMP9 signaling promotes the normalization of tumor blood vessels. Oncogene. 2020; 39: 2996-3014.
74. Vichai V. et al. Sulforhodamine B colorimetric assay for cytotoxicity screening. Nat. Protoc. 2006, 1: 1112-1116.
75. Vinci M. et al. Three-dimensional (3D) tumor spheroid invasion assay. J. Vis. Exp. 2015; e52686.
76. Wakefield L.M. et al. Beyond TGFβ: roles of other TGFβ superfamily members in cancer. Nat. Rev. Cancer. 2013; 13: 328-341.
77. Wang J. et al. Insulin-like growth factor binding protein 5 (IGFBP5) functions as a tumor suppressor in human melanoma cells. Oncotarget. 2015; 6: 20636-20649.
78. Wang X. et al. SOX2 enhances the migration and invasion of ovarian cancer cells via Ste kinase. PLoS One. 2014; 9: e99594.
79. Wegner M. All purpose Sox the many roles of Sox proteins in gene expression. Int. J. Biochem. Cell Biol. 2010; 42: 381-390.
80. Weina K. et al. SOX2 and cancer: current research and its implications in the clinic. Clin. Transl. Med. 2014; 3: 19.
81. Yamamura S. et al. The activated transforming growth factor-beta signaling pathway in peritoneal metastases is a potential therapeutic target in ovarian cancer. Int. J. Cancer. 2012; 130: 20-28.
82. Yang J. et al 5-Aza-2′-deoxycytidine, a DNA methylation inhibitor, induces cytotoxicity, cell cycle dynamics and alters expression of DNA methyltransferase 1 and 3A in mouse hippocampus-derived neuronal HT22 cells. J. Toxicol. Environ. Health. 2017; 80: 1222-1229.
83. Ye L. et al. Bone morphogenetic protein-9 induces apoptosis in prostate cancer cells, the role of prostate apoptosis response-4. Mol. Cancer Res. 2008; 6: 1594-1606.
84. Yeh H.W. et al. PSPC1 mediates TGF-β1 autocrine signalling and Smad2/3 target switching to promote EMT, stemness and metastasis. Nat. Cell Biol. 2018; 20: 479-491.
85. Yuan G.et al. TGF-β signaling inhibits canonical BMP signaling pathway during palate development. Cell Tissue Res. 2018; 371: 283-291.
86. Zeisberg M.et al. BMP-7 counteracts TGF-beta1-induced epithelial-to-mesenchymal transition and reverses chronic renal injury. Nat. Med. 2003; 9: 964-968.
87. Zhang J.et al. Sex-determining region Y-box 2 expression predicts poor prognosis in human ovarian carcinoma. Hum. Pathol. 2012; 43: 1405-1412.
88. Zhang Q. et al. LY2157299 monohydrate, a TGF-βR1 inhibitor, suppresses tumor growth and ascites development in ovarian cancer. Cancers. 2018; 10: 260.
89. Brachova et al. TP53 oncomorphic mutations predict resistance to platinum-and taxane-based standard chemotherapy in patients diagnosed with advanced serous ovarian carcinoma. International journal of oncology, 46, 607-618 (2015).
90. Brouwer-Visser J. et al. Insulin-like growth factor 2 silencing restores taxol sensitivity in drug resistant ovarian cancer. PLoS One, 9, e100165 (2014)
91. Cole et al. Assessing mutant p53 in primary high-grade serous ovarian cancer using immunohistochemistry and massively parallel sequencing. Scientific Reports, 6, 26191 (2016).
92. Dasari et al. Cisplatin in cancer therapy: molecular mechanisms of action. Eur J Pharmacol, 740, 364-78 (2014).
93. Della Pepa et al. Ovarian cancer standard of care: are there real alternatives? Chinese journal of cancer, 34, 17-27 (2015).
94. Hientz, K. et al. The role of p53 in cancer drug resistance and targeted chemotherapy. Oncotarget, 8, 8921-8946 (2017).
95. Housman, G. et al. Drug resistance in cancer: an overview. Cancers, 6, 1769-1792 (2014)
96. Kumari, A. et al. TGFβ signaling networks in ovarian cancer progression and plasticity. Clinical & experimental metastasis, 38, 139-161 (2021).
97. Leiserowitz, G. S. et al. Factors predicting use of neoadjuvant chemotherapy compared with primary debulking surgery in advanced stage ovarian cancer—a national cancer database study. International Journal of Gynecologic Cancer, 27 (2017).
98. Matsuo, K. et al. Overcoming platinum resistance in ovarian carcinoma. Expert opinion on investigational drugs, 19, 1339-1354 (2010).
99. Miller, A. V. et al. Paclitaxel-induced apoptosis is BAK-dependent, but BAX and BIM-independent in breast tumor. PloS one, 8, e60685-e60685 (2013).
100. Rauh-Hain, J. A. et al. Ovarian cancer screening and early detection in the general population. Reviews in obstetrics & gynecology, 4, 15-21 (2011).
101. Sankaranarayanah, R. et al. Worldwide burden of gynaecological cancer: the size of the problem. Best Pract Res Clin Obstet Gynaecol, 20, 207-25 (2006).
102. Shonibare, Z. et al. Reciprocal epigenetic Sox2 regulation by SMAD1-SMAD3 is critical for anoikis resistance and metastasis in cancer. bioRxiv, 2022.01.11.475900 (2022).
103. Vasey, P. A., et al. Phase III randomized trial of docetaxel-carboplatin versus paclitaxel-carboplatin as first-line chemotherapy for ovarian carcinoma. Journal of the National Cancer Institute, 96, 1682-1691 (2004).
104. Vichai, V. et al. Sulforhodamine B colorimetric assay for cytotoxicity screening. Nat Protoc, 1, 1112-6 (2006).
105. Weaver, B. How Taxol/paclitaxel kills cancer cells. Molecular biology of the cell, 25, 2677-2681 (2014).
106. Yoshimatsu, Y. et al. Bone morphogenetic protein-9 inhibits lymphatic vessel formation via activin receptor-like kinase 1 during development and cancer progression. Proc Natl Acad Sci U S A, 110, 18940-5 (2013).
107. Zhang, Y. et al. TP53 mutations in epithelial ovarian cancer. Transl Cancer Res, 5, 650-663 (2016).
108. Shonibere, Z. et al. Reciprocal SOX2 regulation by SMAD1-SMAD3 is critical for anoikis resistance and metastasis in cancer. Cell Reports 40, 111066 (2022).

1. A method for reducing metastasis in a subject with cancer, the method comprising administering an effective amount of BMP9 or an agonist thereof, to the subject with cancer.

2. The method according to claim 1, wherein the BMP9 is recombinant BMP9(rBMP9).

3. The method according to claim 1, wherein the cancer is selected from the group consisting of breast cancer, gynecological cancer, lung cancer, neuroendocrine cancer, and combinations thereof.

4. The method according to claim 1, wherein the cancer is a gynecological cancer.

5. The method according to claim 1, wherein the BMP9 is rhBMP that is administered for a period of at least 7 days.

6. The method according to claim 1, wherein the BMP9 is rhBMP that is administered in an amount from 0.01 mg/kg to 50 mg/kg.

7. The method according to claim 1, wherein the subject is a mammal.

8. The method according to claim 1, wherein the subject is a human.

9. The method according to claim 1, wherein administering the BMP9 or an agonist thereof suppresses three dimensional spheroid cell invasion.

10. The method according to claim 1, wherein the BMP9 or an agonist is administered in a pharmaceutical composition comprising the BMP9 or an agonist and a pharmaceutically acceptable carrier.

11. The method according to claim 10, wherein the pharmaceutical composition further comprises an additional therapeutic agent.

12. The method according to claim 11, wherein the additional therapeutic agent is an anti-cancer compound.

13. The method according to claim 12, wherein the anti-cancer compound is paclitaxel.

14. The method according to claim 12, wherein the anti-cancer compound is cisplatin.
